SEQUENCE LISTING (21 sequences)

SEQ ID NO: 1 - DNA, 20 bp, Artificial Sequence
  chemically synthesized oligonucleotide
gtagcgaacgtgtccggcgt

SEQ ID NO: 2 - DNA, 20 bp, Artificial Sequence
  chemically synthesized oligonucleotide
caggtgctggtggtcaatgg

SEQ ID NO: 3 - DNA, 20 bp, Artificial Sequence
  chemically synthesized oligonucleotide
tctggaaacggaggatgcgg

SEQ ID NO: 4 - DNA, 20 bp, Artificial Sequence
  chemically synthesized oligonucleotide
catacacatcccatatggtg

SEQ ID NO: 5 - DNA, 20 bp, Artificial Sequence
  chemically synthesized oligonucleotide
gcttcaattgcatccttaga

SEQ ID NO: 6 - DNA, 20 bp, Artificial Sequence
  chemically synthesized oligonucleotide
tccgtgaccgtaccatgcag

SEQ ID NO: 7 - DNA, 20 bp, Artificial Sequence
  chemically synthesized oligonucleotide
gccacatctcggagcaactg

SEQ ID NO: 8 - DNA, 20 bp, Artificial Sequence
  chemically synthesized oligonucleotide
gctgctgtagcagcacccca

SEQ ID NO: 9 - DNA, 20 bp, Artificial Sequence
  chemically synthesized oligonucleotide
gatgcggcagccgctgccca

SEQ ID NO: 10 - DNA, 20 bp, Artificial Sequence
  chemically synthesized oligonucleotide
gtcaagcagcggcggagctt

SEQ ID NO: 11 - DNA, 20 bp, Artificial Sequence
  chemically synthesized oligonucleotide
gcagcatccaaccaaaatcc

SEQ ID NO: 12 - DNA, 20 bp, Artificial Sequence
  chemically synthesized oligonucleotide
gcttgaagggtctgacgctt

SEQ ID NO: 13 - DNA, 20 bp, Artificial Sequence
  chemically synthesized oligonucleotide
ggatcttctcgccctcagag

SEQ ID NO: 14 - DNA, 20 bp, Artificial Sequence
  chemically synthesized oligonucleotide
catgaagttccagtacaagg

SEQ ID NO: 15 - DNA, 20 bp, Artificial Sequence
  chemically synthesized oligonucleotide
ttcccgccgccgccatgaag

SEQ ID NO: 16 - DNA, 20 bp, Artificial Sequence
  chemically synthesized oligonucleotide
tcacgccattcctacaccag

SEQ ID NO: 17 - DNA, 20 bp, Artificial Sequence
  chemically synthesized oligonucleotide
caacatgtccacactctgag

SEQ ID NO: 18 - DNA, 20 bp, Artificial Sequence
  chemically synthesized oligonucleotide
ccagttgatatgccaagcag

SEQ ID NO: 19 - RNA, 42 nt, Artificial Sequence
  chemically synthesized oligonucleotide
guuaggggccaucacggcucguuuuagagcuaugcuguuuug

SEQ ID NO: 20 - DNA, 119 bp, Artificial Sequence
  chemically synthesized oligonucleotide
gctgtctcccttaggtcagagagagtgtggtccgagagatggaactgttaggggcgatcaccgctttggacctaaaccctgagagaactgagctcctgagctgctcccgtgatgacctg

SEQ ID NO: 21 - DNA, 20 bp, Artificial Sequence
  chemically synthesized oligonucleotide
gtggatactcatcctggttc

(19) United States
(12) Patent Application Publication
(10) Pub. No.: US 2023/0272392 A1
(43) Pub. Date: Aug. 31, 2023

(54) METHODS OF TFEB ACTIVATION AND LYSOSOMAL BIOGENESIS AND COMPOSITIONS THEREFOR

(71) Applicant: Casma Therapeutics, Inc., Cambridge, MA (US)

(72) Inventors: Jonathan Michael Goodwin, Beverly, MA (US); Oliver Florey, Cambridgeshire (GB); Leon Murphy, Newton, MA (US)

(21) Appl. No.: 18/019,275

(22) PCT Filed: Jul. 26, 2021
(86) PCT No.: PCT/US21/43125

§ 371 Date: Feb. 2, 2023

Related U.S. Application Data
(60) Provisional application No. 63/150,520, filed on Feb. 17, 2021.
(60) Provisional application No. 63/060,611, filed on Aug. 3, 2020.

(51) Int. Cl. C12N 15/113; A61K 45/06; A61P 3/00

(52) U.S. Cl. CPC C12N 15/113; A61K 45/06; A61P 3/00

(57) ABSTRACT

The present disclosure pertains to methods of activating TFEB independent of mTORC1 activity, methods of activating TFEB by enhancing GABARAP/FNIP/FLCN complex localization at an intracellular membrane surface, methods of characterizing a TFEB activating agent, and methods of treating a TRPML1-associated disease, disorder or condition, and compositions for use in said methods.

### CROSS-REFERENCE TO RELATED APPLICATIONS

The present application claims priority to U.S. Provisional Application No. 63/060,611, filed Aug. 3, 2020, and U.S. Provisional Application No. 63/150,520, filed Feb. 17, 2021, which are incorporated herein in their entirety.

### SEQUENCE LISTING

In accordance with 37 CFR 1.52(e)(5), a Sequence Listing in the form of an ASCII text file (entitled “2013075-0042_SL.txt”, created on Jul. 22, 2021, and 4,821 bytes in size) is incorporated herein by reference in its entirety.

### BACKGROUND

Ion homeostasis and acidic pH are tightly coupled determinants of lysosomal degradative capacity and alterations in these properties can cause disease. Although lysosomal membrane channels are known to regulate ion flux, how such changes are sensed locally by the lysosome and how the cell responds is unclear.

### SUMMARY

The present disclosure provides, among other things, an insight that stimulating, stabilizing, localizing, and/or otherwise increasing the membrane localization of the GABARAP/FLCN/FNIP complex through lipidation and subsequent conjugation of GABARAP proteins (GABARAP, GABARAPL1, GABARAPL2) may activate TFEB independent of mTORC1 activity and/or may otherwise increase autophagy.

Alternatively or additionally, in some embodiments, the present disclosure provides an insight that agonizing TRPML1 can stimulate, stabilize, localize, and/or otherwise increase levels of a GABARAP/FLCN/FNIP complex at cytosolic surfaces positive for LAMP1 (e.g., lysosomal membrane surfaces).

The present disclosure also provides technologies for assessing agents that increase autophagy and/or that agonize TRPML1 and/or that stimulate, stabilize, localize, and/or otherwise increase levels of a GABARAP/FLCN/FNIP complex at cytosolic surfaces positive for LAMP1 (e.g., lysosomal membrane surfaces); still further, the present disclosure provides an insight that such agents may be useful in the treatment of certain diseases, disorders or conditions, including those that may be associated with defects in the conjugation machinery responsible for GABARAP membrane localization and/or that might benefit from increased autophagy.

In one aspect, the present disclosure provides methods of activating TFEB independent of mTORC1 activity comprising a step of contacting a system that comprises a membrane comprising LAMP-1, vATPase or GABARAP and components of a GABARAP/FLCN/FNIP complex with a TRPML1 agonist such that level of the GABARAP/FLCN/FNIP complex at the membrane is elevated. In some embodiments, a membrane comprising LAMP-1 vATPase or GABARAP defines a compartment. In some embodiments, a compartment is or comprises a lysosome. In some embodiments, a membrane is or comprises a lysosomal membrane. In some embodiments, a lysosomal membrane is part of an intact lysosome. In some embodiments, a lysosome is in a cell.

In another aspect, the present disclosure provides methods of activating TFEB independent of mTORC1 activity comprising a step of administering a TRPML1 agonist. In some embodiments, a step of administering comprises contacting a system with the TRPML1 agonist, wherein the system comprises a lysosomal membrane and components of a GABARAP/FLCN/FNIP complex.

In some embodiments, a system has a polymorphism or mutation in a gene encoding a conjugation machinery protein (conjugation machinery gene) and/or a gene encoding a component of the GABARAP/FLCN/FNIP complex. In some embodiments, a conjugation machinery gene is selected from the group consisting of Atg3, Atg5, Atg7, Atg12, Atg16L1, and combinations thereof. In some embodiments, a conjugation pathway gene is Atg16L1. In some embodiments, a polymorphism is T300A.

In some embodiments, a TRPML1 agonist is of a chemical class selected from the group consisting of polypeptides, nucleic acids, lipids, carbohydrates, small molecules, metals, and combinations thereof.

In some embodiments, a step of administering comprises exposing the system to the TRPML1 agonist under conditions and for a time sufficient that enhanced expression or activity of one or more CLEAR network genes and/or enhancement of one or more of detectable exocytosis activity, autophagy, clearance of lysosomal storage material, and lysosomal biogenesis is observed in the system relative to that prior to the exposure. In some embodiments, a step of administering comprises exposing the system to the TRPML1 agonist under conditions and for a time sufficient that enhanced expression or activity of one or more genes selected from Table 1 is observed in the system relative to that prior to the exposure.

In some embodiments, a TRPML1 agonist is characterized in that, when assessed for impact on expression of CLEAR network genes, it shows a more restricted impact than that observed under starvation conditions. In some embodiments, a TRPML1 agonist is characterized in that TRPML1 level or activity is higher in its presence than in its absence, under comparable conditions.

In some embodiments, a TRPML1 agonist is a direct agonist in that it interacts with TRPML1. In some embodiments, a TRPML1 agonist is an indirect agonist in that it does not directly interact with TRPML1.

In another aspect, the present disclosure provides methods of treating a TRPML1-associated disease, disorder or condition comprising a step of administering a TRPML1 agonist to a subject suffering from, or susceptible to, the TRPML1-associated disease, disorder or condition. In some embodiments, a TRPML1-associated disease, disorder or condition is or comprises an inflammatory condition. In some embodiments, a TRPML1-associated disease, disorder or condition is or comprises a lysosomal storage disorder. In some embodiments, a TRPML1-associated disease, disorder or condition is or comprises a polyglutamine disorder. In some embodiments, a TRPML1-associated disease, disorder or condition is or comprises a neurodegenerative proteinopathy. In some embodiments, a TRPML1-associated disease, disorder or condition is an infectious disease. In some embodiments, a TRPML1-associated disease, disorder or condition is selected from a group consisting of Crohn's Disease, Pompe Disease, Parkinson's Disease, Huntington's Disease, Alzheimer's Disease, Spinal-bulbar muscular atrophy, α-1-antitrypsin deficiency, and multiple sulfatase deficiency. In some embodiments, a TRPML1-associated disease, disorder or condition is Crohn's Disease.

In another aspect, the present disclosure provides methods of activating TFEB by enhancing GABARAP/FNIP/FLCN complex localization at an intracellular membrane surface. In some embodiments, an intracellular membrane surface is a cytosolic surface of an intracellular compartment. In some embodiments, an intracellular compartment is a lysosome. In some embodiments, an intracellular compartment is a mitochondria. In some embodiments, an intracellular compartment is an endoplasmic reticulum. In some embodiments, an intracellular compartment is a pathogen vacuole. In some embodiments, an intracellular compartment is an endosomal structure.

In some embodiments, a method comprises administering a TRPML1 agonist. In some embodiments, TFEB activation is independent of mTORC1 activity.

In another aspect, the present disclosure provides methods of characterizing a TFEB activating agent, the methods comprising assessing effect on FLCN localization and/or level of a GABARAP/FNIP/FLCN complex at one or more intracellular membrane surfaces.

In another aspect, the present disclosure provides methods of treating a conjugation-machinery-associated (“CMA”) disease, disorder or condition or a GABARAP/FNIP/FLCN complex-associated disease, disorder or condition, the methods comprising a step of administering a TRPML1 agonist. In some embodiments, a disease, disorder or condition is or comprises Crohn's Disease.

In another aspect, the present disclosure provides methods comprising a cellular assay for characterizing activators of TFEB, TFE3 and/or MITF, wherein the cellular assay comprises cells comprising presence of a vATPase small molecule inhibitor, genetic disruption of ATG8 conjugation machinery, presence of a small molecule inhibitor of ATG8 conjugation machinery, genetic disruption of a member of a GABARAP subfamily of proteins; mutation of a LIR domain in FNIP1 or FNIP2, or a combination thereof. In some embodiments, a vATPase small molecule inhibitor is Bafilomycin A1. In some embodiments, a vATPase small molecule inhibitor is not an analogue of Salicylihalamide A. In some embodiments, a genetic disruption of ATG8 conjugation machinery comprises knock-out of a gene, knock-in of a gene, expression of one or more mutant alleles, siRNA, shRNA, antisense, or a combination thereof. In some embodiments, a genetic disruption of the member of a GABARAP subfamily of proteins comprises knock-out of a gene, knock-in of a gene, expression of one or more mutant alleles, siRNA, shRNA, antisense, or a combination thereof.

### BRIEF DESCRIPTION OF THE DRAWING

The drawings are for illustration purposes only, not for limitation.

FIG. 1 shows a Western blot illustrating an exemplary effect of different treatments on LC3 lipidation.

FIG. 2 shows cell images illustrating an exemplary effect of different treatments on LC3 lipidation.

FIG. 3 shows a Western blot illustrating an exemplary effect of different treatments on LC3 lipidation.

FIG. 4 shows a Western blot illustrating an exemplary effect of AZD8055, EBSS starvation, and Bafilomycin A1 (BafA1) on LC3 lipidation.

FIG. 5 shows a Western blot illustrating an exemplary effect of knocking out TRPML1 (MCOLN1) on LC3 lipidation.

FIG. 6 shows cell images illustrating that L3C lipidation following treatment was not accompanied by lysosomal alkalization or membrane damage.

FIG. 7 shows cell images illustrating co-localization of ATG8s (LC3B or GABARAPL1) with the lysosomal marker LAMP1 after treatment with the TRPML1 agonist C8.

FIG. 8 shows graphs illustrating co-localization of ATG8s (LC3B or GABARAPL1) with the lysosomal marker LAMP1 after treatment with the TRPML1 agonist C8.

FIG. 9 shows cell images illustrating that TRPML1 agonist (e.g., C8)-induced LC3 puncta formation was abolished upon introduction of the ATG16L1 mutant K490A in ATG16L1 KO cells.

FIG. 10 shows images from ultrastructural correlative light electron microscopy (CLEM) illustrating GFP-LC3 structures characteristic of lysosomes following treatment with C8 or AZD8055.

FIG. 11 shows a diagram and a Western blot illustrating Salmonella SopF impairment of ATG16L1 recruitment by the vATPase and effect of SopF-induction on LC3-II formation after treatment with C8 or AZD8055.

FIG. 12 shows cell images illustrating the effect of treatment with C8 or ML-SA1 on LC3 puncta formation co-localized with LAMP1 in wild-type cells or ATG16L1^{K490A} cells.

FIG. 13 shows a graph illustrating involvement of calcineurin on the effect of TRPML1 agonists on TFEB activation.

FIG. 14 shows graphs illustrating involvement of calcineurin on the effect of TRPML1 agonists on TFEB activation.

FIG. 15 shows a graph illustrating an effect of SopF expression in cells treated with a TRPML1 agonist on TFEB nuclear accumulation.

FIG. 16 shows a Western blot illustrating effects of BafA1 and AZD8055 on TFEB activation by TRPML1 agonists (MK6-83 and C8).

FIG. 17 shows a Western blot illustrating effects of knocking out FIP200, ATG9A, VPS34, ATG5, ATG7, or ATG16L1 on TFEB activation and LC3 conjugation.

FIG. 18 shows a Western blot illustrating effects knockout of ATG16L1, co-treatment with BafA1, or nutrient starvation (EBSS) on TFEB activation by a TRPML1 agonist (C8).

FIG. 19 shows graphs illustrating effects of drugs that display ionophore properties and regulate single-membrane ATG8 conjugation on TFEB activation.

FIG. 20 shows a Western blot illustrating effects of several ATG16L1 alleles including a FIP200 binding mutant (ΔFBD) and a C-terminal domain truncation (ΔCTD) on TFEB expression in ATG16L1 knockout cells treated with a TRPML1 agonist (C8).

FIG. 21 shows cell images illustrating that ATG16L1 KO mouse macrophages reconstituted with WD40 point mutations ATG16L1-F467A (F467A) and ATG16L1-K490A (K490A) did not exhibit TFEB activation in the presence of TRPML1 agonists (e.g., C8 and ML-SA1).

FIG. 22 shows a graph illustrating expression of target genes in control and ATG16L1 knockout cells after treatment with a TRPML1 agonist.

FIG. 23 shows a heat map illustrating a transcriptomic response in wild-type and ATG16L1 knock out cells after treatment with a TRPML1 agonist.

FIG. 24 shows a heat map illustrating a transcriptomic response in wild-type and ATG16L1 knock out cells after treatment with a TRPML1 agonist.

FIG. 25 shows cell images in panel A and graphs in panels B and C illustrating the effect of TRPML1 activation on the number and intensity of Lysotracker-positive organelles.

FIG. 26 shows a Western blot and a graph illustrating the involvement of GABARAP proteins in the activation of TFEB upon treatment with a TRPML1 agonist.

FIG. 27 shows a Western blot illustrating co-immunoprecipitation of GABARAP with the FLCN-FNIP complex.

FIG. 28 shows a Western blot illustrating the effect of a GABARAP protein comprising a mutation in the LIR domain docking site (LDS) of GABARAP, on pulling down the FLCN-FNIP complex.

FIG. 29 shows a diagram illustrating the direct conjugation of GABARAPs to lysosomal membranes in response to changes in lysosomal flux and the effect on the FLCN/FNIP complex.

FIG. 30 shows a Western blot illustrating an increase in membrane-associated FLCN and FNIP1 following TRPML1 activation.

FIG. 31 shows cell images illustrating the effect of a TRPML1 agonist on co-localization of FLCN with the lysosomal protein LAMP1.

FIG. 32 shows a Western blot illustrating recruitment of FLCN and FNIP1 after treatment with a TRPML1 agonist.

FIG. 33 shows a Western blot illustrating FLCN and FNIP1 after treatment with a TRPML1 agonist in cells deficient for the Ragulator complex component LAMTOR1.

FIG. 34 shows cell images and a Western blot illustrating the effect of knocking out FLCN on nuclear translocation of TFEB under nutrient rich conditions in both wild-type and NPRL2 KO cells.

FIG. 35 shows a Western blot illustrating the effect of RagGTPases locked in the active state on TFEB following TRPML1 activation.

FIG. 36 shows a Western blot illustrating the effect of non-TRPML1 stimuli on GABARAP-dependent sequestration of FLCN.

FIG. 37 shows a diagram illustrating a cellular mechanism of TFEB activation upon alteration of lysosomal ion contents.

FIG. 38 shows a graph illustrating co-purification of GABARAP and the FLCN-FNIP2 complex in a sizing column.

FIG. 39 shows a graph and a protein structure model illustrating a binding site between GABARAP and FLCN-FNIP2.

FIG. 40 shows a Western blot illustrating the involvement of a GABARAP LIR domain in interaction with the FLCN-FNIP1 complex.

FIG. 41 shows a Western blot illustrating the effect of mutations in the FNIP1 LIR on the interaction between FNIP1 and FLCN.

FIG. 42 shows a Western blot and cell images illustrating TFEB and TFE3 activation in FNIP1/FNIP2 double knockout cells.

FIG. 43A shows TFEB activation in parental, FNIP1/FNIP2 double knockout, FNIP1/FNIP2 double knockout with wild-type FNIP1 and FNIP1/FNIP2 double knockout and LIRmut-FNIP1 cells treated with DMSO, starvation or a TRPML1 agonist.

FIG. 43B shows a Western blot illustrating TFEB activation in parental, FNIP1/FNIP2 double knockout, FNIP1/FNIP2 double knockout with wild-type FNIP1 and FNIP1/FNIP2 double knockout and LIRmut-FNIP1 cells treated with DMSO, starvation or a TRPML1 agonist.

FIG. 44A shows graphs illustrating TFEB activation in parental, FNIP1/FNIP2 double knockout, FNIP1/FNIP2 double knockout with wild-type FNIP1 and FNIP1/FNIP2 double knockout and LIRmut-FNIP1 cells treated with DMSO, starvation or a TRPML1 agonist.

FIG. 44B shows graphs illustrating TFEB activation in FNIP1/FNIP2 double knockout with wild-type FNIP1 and FNIP1/FNIP2 double knockout and LIRmut-FNIP1 cells treated with DMSO, starvation or a TRPML1 agonist.

FIG. 44C shows a Western blot illustrating that the FNIP1-LIR domain is involved in membrane localization of the FLCN-FNIP complex upon treatment with a TRPML1 agonist.

FIG. 44D shows a Western blot illustrating elevation of the TFEB target gene GPNMB at the protein level upon treatment with a TRPML1 agonist in comparison to the mTOR inhibitor AZD8055. Upregulation of GPNMB is dependent on the FNIP1-LIR domain.

FIG. 45 shows a graph illustrating TFEB intensity in HeLa.Cas9 or HeLa.Cas9+Parkin cells treated with the indicated compounds (DMSO, valinomycin or oligomycin/antimycin (O/A)) for 4 hours.

FIG. 46 shows a Western blot illustrating the effect of valinomycin on HeLa control knock-out, LC3 triple knock-out or GABARAP triple knock-out cells expressing Parkin, which is needed for robust TFEB activation upon stimulation of mitophagy.

FIG. 47 shows a Western blot illustrating TFEB activation in FNIP1/FNIP2 double knockout cells stably expressing LIR-mutant FNIP1 after treatment with mitophagy inducers (valinomycin and OA) or control (DMSO) for 24 hours.

FIG. 48 shows an illustration of a proximity-based mitophagy induction model. Recruitment of p62 to mitochondria results in mitophagy, independent of chemical disruption of mitochondrial function.

FIG. 49 shows a graph illustrating quantification of mitophagy efficiency in U2OS cells expressing mKeima, FRB-p62, and FKBP-FIS1 and that co-treatment with BafA1 blocked the Keima signal.

FIG. 50 shows TFE3 and FLCN subcellular localization in U2OS cells upon dimerizer-induced mitophagy. Cells of the indicated genotype (control knock-out, RB1 CC1 knock-out and GABARAP triple knock-out) were stimulated with 25 nM AP21967 (dimerizer) for 3 hours. TFE3 nuclear localization and FLCN punctate structures were seen in CTRL and RBICC1_KO (autophagy-deficient) cells but not in GABARAP_TKO cells.

FIG. 51 shows a Western blot illustrating a TFEB mobility shift upon challenge with wild-type (WT) or ΔsopF Salmonella. HeLa cells were infected for 30 minutes with the indicated strain and lysates were taken at the indicated time points post-infection.

FIG. 52 shows immunofluorescence analysis of nuclear TFEB accumulation upon Salmonella infection. Cells were infected with wild-type (WT) or ΔsopF Salmonella and analyzed at 2 hours post infection (h.p.i.).

FIG. 53 shows a graph illustrating quantified TFEB nuclear localization in cells infected with wild-type (WT) or ΔsopF Salmonella. A minimum of 100 cells were quantified per condition.

FIG. 54 shows immunofluorescence analysis of TFEB expression in control knock-out, LC3 triple knock-out or GABARAP triple knock-out cells infected with ΔsopF Salmonella and analyzed at 2 hours post infection (h.p.i.).

FIG. 55 shows a graph illustrating quantified TFEB nuclear localization in wild-type, LC3 triple knock-out and GABARAP triple knock-out cells.

FIG. 56 shows graphs illustrating TFEB transcriptional activity in cells of the indicated genotype (wild-type, LC3 triple knock-out and GABARAP triple knock-out) at 10 hours post infection (h.p.i) with wild-type (control) or ΔsopF Salmonella. GPNMB and RRAGD were used as core TFEB target genes.

FIG. 57 shows immunofluorescence analysis of FLCN recruitment to Salmonella vacuoles in control knock-out, LC3 triple knock-out and GABARAP triple knock-out cells infected with S. Typhimurium Salmonella. Deletion of GABARAP proteins (RAP_TKO), but not LC3 family members (LC3_TKO), blocked the re-localization of FLCN.

FIG. 58 shows an illustration of GABARAP-dependent membrane sequestration of the FLCN-FNIP complex as a TFEB activation paradigm distinct from nutrient starvation.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Agonist: As will be understood by those skilled in the art, the term “agonist” generally refers to an agent whose presence or level correlates with elevated level or activity of a target, as compared with that observed absent the agent (or with the agent at a different level). In some embodiments, an agonist is one whose presence or level correlates with a target level or activity that is comparable to or greater than a particular reference level or activity (e.g., that observed under appropriate reference conditions, such as presence of a known agonist, e.g., a positive control). In some embodiments, an agonist may be a direct agonist in that it exerts its influence directly on (e.g., interacts directly with) the target; in some embodiments, an agonist may be an indirect agonist in that it exerts its influence indirectly (e.g., by acting on, such as interacting with, a regulator of the target, or with some other component or entity.

Antagonist: As will be understood by those skilled in the art, the term “antagonist” generally refers to an agent whose presence or level correlates with decreased level or activity of a target, as compared with that observed absent the agent (or with the agent at a different level). In some embodiments, an antagonist is one whose presence or level correlates with a target level or activity that is comparable to or less than a particular reference level or activity (e.g., that observed under appropriate reference conditions, such as presence of a known antagonist, e.g., a positive control). In some embodiments, an antagonist may be a direct antagonist in that it exerts its influence directly on (e.g., interacts directly with) the target; in some embodiments, an antagonist may be an indirect antagonist in that it exerts its influence indirectly (e.g., by acting on, such as interacting with, a regulator of the target, or with some other component or entity.

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalveolar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### DETAILED DESCRIPTION OF CERTAIN EMBODIMENTS

The present disclosure provides the insights of a mechanism whereby small molecule agonists of the lysosomal ion channel TRPML1 promote rapid and robust conjugation of ATG8 proteins directly to the lysosomal surface. Conjugation of GABARAP proteins, specifically, results in membrane sequestration of the FLCN-FNIP complex away from its substrate RagC/RagD. This results in RagC/RagD remaining in the “GTP-bound” state and impaired binding to TFEB/TFE3/MITF transcription factors. As RagC/RagD in the “GDP-bound” state promotes cytoplasmic retention, the increased “GTP-bound” RagC/RagD promotes TFEB/TFE3/MITF nuclear localization.

### Autophagy, Lysosomes and TFEB

Autophagy is an evolutionarily conserved cellular process that allows for the breakdown and recycling of cytoplasmic components, termed “cargo”. This cargo can range from single proteins to protein aggregates; from organelles to invading pathogens. This process involves encapsulating cargo in a double membrane autophagosome and eventual fusion with a lysosome (1). Lysosomes are single membrane organelles that contain acidic hydrolases and peptidases that break down cargo to single amino acids. In addition to its critical degradative function, the lysosome is a major signaling platform within the cell. Information about the abundance of nutrients, including, but not limited to, amino acids, lipids, and sugars, can be communicated through various lysosomal resident proteins and signaling complexes (33).

The formation of lysosomes and the transcriptional regulation of numerous lysosomal enzymes, membrane proteins, and autophagy components are under the control of the master transcription factors TFE3 and TFEB. Under conditions of nutrient stress or increased autophagic flux, these transcription factors localize to the nucleus and orchestrate a transcriptional program driving lysosomal biogenesis (15).

A major regulator of TFE3 and TFEB nuclear localization is the mTORC1 complex. This signaling complex resides on the lysosomal surface and senses cellular nutrient status. mTOR, a component of mTORC1, phosphorylates TFE3 and TFEB in response to nutrients to facilitate cytoplasmic retention. When nutrients are low, mTORC1 becomes inactivated and the repression of TFE3 and TFEB nuclear localization is relieved (34).

Independent from mTORC1 phosphorylation, TFE3 and TFEB transcription factors can be activated by changes in lysosomal ion contents. This has been demonstrated with small molecule agonists of the transient receptor potential ML1 (TRPML1) ion channel. Agonist binding triggers non-selective release of positive cations from the lysosomal lumen to the cytosol. Previous work has shown that the release of lysosomal calcium is required for TRPML1 agonist activation of TFE3 and TFEB (14).

Perhaps the most dominant regulator of TFE3 and TFEB nuclear localization is the nucleotide state of the small GTPases RagC/RagD (24). When these small GTPases are in the “GTP-bound” state, they cannot bind to TFE3 and TFEB and result in nuclear accumulation. RagGTPases are well documented sensors of amino acid levels in a cell and deprivation of amino acids (starvation) promotes the RagC/RagD “GTP-bound state”. Upon stimulation with amino acids, a complex consisting of the tumor suppressor FLCN and its binding partner FNIP1 or FNPI2 acts as a GAP (GTPase-activating protein) and triggers RagC/RagD GTP-hydrolysis to result in a “GDP-bound state”. This “GDP-bound state” is permissive for direct interaction of RagC/RagD with TFE3 and TFEB and promotes the cytosolic retention of these transcription factors. In support of this regulation, expression of a constitutively “GTP-bound” form of RagC can result in constitutive nuclear localization of TFE3 and TFEB in the presence of nutrients, while allowing for proper access of mTORC1 to other substrates involved in anabolic growth processes. Conversely, expression of “GDP-bound” RagC can suppress the nuclear accumulation of TFE3 and TFEB in starved conditions (35).

The present disclosure highlights a novel mechanism for the regulation of TFEB and TFE3 transcriptional activity. Using small molecule agonists of the lysosomal ion channel TRPML1, a novel pathway of single membrane ATG8 conjugation (SMAC) has been uncovered. Changes in lysosomal ion balance trigger a compensatory response from the vacuolar ATPase (vATPase), whereby the ATG5-ATG12-ATG16L1 complex is directly recruited to the lysosomal membrane and conjugates ATG8 homologs of the LC3 and GABARAP subfamilies to the cytosolic surface of lysosomes. Specifically, the conjugation of GABARAP proteins to the lysosomal membrane results in a sequestration of the GABARAP-bound FLCN-FNIP complex. When robustly localized to the lysosomal membrane, and in some embodiments other membranes, the FLCN-FNIP complex is restricted from acting on its substrate RagC/RagD. The regulation of RagC/RagD by FLCN-FNIP normally occurs in the cytosol as opposed to the lysosomal membrane as proposed previously. Without GAP activity provided by FLCN-FNIP, RagC/RagD remain in the GTP-bound state and promote TFEB and TFE3 nuclear accumulation. This process is independent from regulation of mTORC1 activity and independent from a previously proposed mechanism of calcineurin (CaN)-mediated TFEB and TFE3 dephosphorylation. In some embodiments, GABARAP-dependent sequestration of the FLCN-FNIP complex can occur with stimuli other than a TRPML1 agonist. In some embodiments, GABARAP conjugation to intracellular membranes including, but not limited to, autophagosomes, mitochondria, pathogen containing vacuoles, endoplasmic reticulum, the plasma membrane, endosomes, and multivesicular bodies can activate TFEB/TFE3 via GABARAP-dependent sequestration of the FLCN-FNIP complex.

### Active Agents

In some embodiments, the present disclosure provides agents that increase autophagy and/or that agonize TRPML1 and/or that stimulate, stabilize, localize, and/or otherwise increase level of a GABARAP/FLCN/FNIP complex at membrane surfaces (e.g., lysosomal surfaces, cytosolic surfaces, or surfaces in association with LAMP1) and/or methods of making, characterizing, and/or using said agents. In some embodiments, an agent of the present disclosure is or comprises a modulator selected from the group consisting of polypeptides, nucleic acids, lipids, carbohydrates, small molecules, metals, and combinations thereof.

In some embodiments, an agent of the present disclosure is or comprises an agent that exhibit lysosomotropic and ionophore/protonophore-like properties. In some embodiments, an agent is an inhibitor of mitochondrial ATP synthase. In some embodiments, an agent is an inhibitor of cytochrome C reductase. In some embodiments, an agent is selected from a group consisting of monensin, nigericin, salinomycin, valinomycin, oligomycin, antimycin, chloroquine, and CCCP.

In some embodiments, the present disclosure provides agents that are or comprise TRPML1 modulators of a chemical class selected from the group consisting of polypeptides, nucleic acids, lipids, carbohydrates, small molecules, metals, and combinations thereof. In some embodiments, TRPML1 modulators are small molecule compounds. In some embodiments, a TRPML1 modulator comprises ML-SA1, ML-SA3, ML-SA5, MK6-83, C8 (see WO 2018/005713), or C2 (see WO 2018/005713). In some embodiments, a TRPML1 modulator may show activity in one or more assays as described herein. In some embodiments, a small molecule compound is determined to be a TRPML1 modulator by showing activity in a TFEB assay wherein TFEB translocation is measured after wild-type and TRPML1 knock-out HeLa cells are treated with the small molecule compound. In some embodiments, a small molecule compound is determined to be a TRPML1 modulator by showing endogenous lysosomal calcium flux activity in an assay comprising Fluorescent Imaging Plate Reader (FLIPR) technology performed on wild-type and TRPML1 knock-out HeLa cells treated with the small molecule compound. In some embodiments, a small molecule compound is determined to be a TRPML1 modulator by showing exogenous calcium flux activity in an assay comprising Fluorescent Imaging Plate Reader (FLIPR) technology performed on a cell line that expresses tetracycline-inducible TRPML1 on the cell surface and has been treated with the small molecule compound.

In some embodiments, a TRPML1 modulator is a TRPML1 agonist. In some embodiments, a TRPML1 agonist is characterized in that, when assessed for impact on expression of CLEAR network genes, it shows a more restricted impact than that observed under starvation conditions. In some embodiments, a TRPML1 agonist is characterized in that TRPML1 level or activity is higher in its presence than in its absence, under comparable conditions. In some embodiments, a TRPML1 agonist is a direct agonist in that it interacts with TRPML1. In some embodiments, a TRPML1 agonist is an indirect agonist in that it does not directly interact with TRPML1.

### Compositions

In some embodiments, the present disclosure provides compositions that comprise and/or deliver an active agent as described herein.

### Uses

In some embodiments, the present disclosure provides technologies for using described agents, for example to activate TFEB independent of mTORC1; to stimulate, stabilize, localize, and/or otherwise increase level of a GABARAP/FLCN/FNIP complex at one or more membrane surfaces (e.g., cytosolic surfaces, e.g., in association with LAMP1; e.g., of a lysosome); to treat diseases benefitting from increased lysosomal biogenesis and/or increased lysosomal enzyme activity and/or increased mitochondrial biogenesis.

In some embodiments, the present disclosure provides a method of activating TFEB independent of mTORC1 activity, the method comprising a step of contacting a system that comprises a membrane comprising LAMP-1, vATPase or GABARAP; and components of a GABARAP/FLCN/FNIP complex; with a TRPML1 agonist such that level of the GABARAP/FLCN/FNIP complex at the membrane is elevated. In some embodiments, a membrane comprising LAMP-1 vATPase or GABARAP defines a compartment. In some embodiments, a compartment is or comprises a lysosome. In some embodiments, a compartment is or comprises a late endosome. In some embodiments, a compartment is or comprises a multivesicular body. In some embodiments, a membrane is or comprises a lysosomal membrane. In some embodiments, a membrane is or comprises an endosomal membrane. In some embodiments, a membrane is or comprises a multivesicular body membrane. In some embodiments, a lysosomal membrane is part of an intact lysosome. In some embodiments, an endosomal membrane is part of an intact endosome. In some embodiments, a multivesicular body membrane is part of an intact multivesicular body. In some embodiments, a lysosome is in a cell. In some embodiments, an endosome is in a cell. In some embodiments, a multivesicular body is in a cell.

In some embodiments, the present disclosure provides a method of activating TFEB independent of mTORC1 activity, the method comprising a step of administering a TRPML1 agonist. In some embodiments, the step of administering comprises contacting a system with a TRPML1 agonist, wherein the system comprises a lysosomal membrane and components of a GABARAP/FLCN/FNIP complex. In some embodiments, a system has a polymorphism or mutation in a gene encoding a conjugation machinery protein (conjugation machinery gene) and/or a gene encoding a component of the GABARAP/FLCN/FNIP complex. In some embodiments, a conjugation machinery gene is selected from the group consisting of Atg3, Atg5, Atg7, Atg12, Atg16L1, and combinations thereof. In some embodiments, a conjugation pathway gene is Atg16L1. In some embodiments, a polymorphism is T300A. In some embodiments, a TRPML1 agonist is of a chemical class selected from the group consisting of polypeptides, nucleic acids, lipids, carbohydrates, small molecules, metals, and combinations thereof.

In some embodiments, a the step of administering comprises exposing a system to a TRPML1 agonist under conditions and for a time sufficient that enhanced expression or activity of one or more Coordinated Lysosomal Expression and Regulation (CLEAR) network genes and/or enhancement of one or more of detectable exocytosis activity, autophagy, clearance of lysosomal storage material, and lysosomal biogenesis is observed in the system relative to that prior to the exposure. In some embodiments, CLEAR network genes are targeted and/or controlled by TFEB. In some embodiments, CLEAR network genes are involved in regulating the expression, import and activity of lysosomal enzymes that control the degradation of proteins, glycosaminoglycans, sphingolipids and glycogen. In some embodiments, CLEAR network genes are involved in the regulation of additional lysosome-associated processes, including autophagy, exocytosis, endocytosis, phagocytosis and immune response. In some embodiments, CLEAR network genes comprise genes encoding non-lysosomal enzymes involved in the degradation of essential proteins such as hemoglobin and chitin.

In some embodiments, a TRPML1 agonist is characterized in that, when assessed for impact on expression of CLEAR network genes, it shows a more restricted impact than that observed under starvation conditions. In some embodiments, a TRPML1 agonist is characterized in that, when assessed for impact on expression of CLEAR network genes, it does not show a more restricted impact than that observed under starvation conditions.

In some embodiments, a step of administering comprises exposing a system to a TRPML1 agonist under conditions and for a time sufficient that enhanced expression or activity of one or more genes selected from Table 1 is observed in the system relative to that prior to the exposure.

TABLE 1

ABHD3
ABLIM2
ACAT2
ACSF2
ACSS2
ADM
ADM2
AHNAK2
AK7
ALDOC
ALOX12
ALPK1
AMDHD2
ANKRD1
ANO3
AP5Z1
APLN
ARC
ARHGAP11B
ARHGAP12
ARHGAP20
ARHGAP31
ARHGEF35
ARHGEF4
ARL4C
ARVCF
ASB2
ATF3
ATP2A1
ATP6V0E2-AS1
ATP6V1C1
AVPI1
B3GNT5
BEX2
BHLHE40
BHLHE40-AS1
BHLHE41
BORCS6
BRI3
BTBD19
C2orf16
C6orf223
C7orf57
CASC19
CASP4
CCDC171
CCDC24
CD180
CD70
CDC25B
CDKN1A
CEMIP
CHAC1
CHKA
CHN2
CHRDL2
CLCN5
CLCN6
CLCN7
CLDN23
CLIC2
CLIP2
CNNM2
CNTF
CPA4
CPEB4
CREBRF
CSF2RA
CTNS
CYB5B
CYP24A1
CYP51A1
DAPK2
DCLK1
DDIT3
DERL3
DHCR24
DHCR7
DHRS3
DOK7
DTNA
DTX4
DUSP1
DUSP10
DUSP3
DUSP4
DUSP5
EDNRA
EEPD1
ENC1
ENO2
EREG
ERG28
ERN1
ERRFI1
ETV1
ETV5
F2R
F2RL1
FABP3
FADS2
FAM131C
FAM49A
FAM72A
FAM72B
FAM72C
FAM78A
FBXO10
FBXO32
FCMR
FDFT1
FGFR1
FHL2
FLCN
FLI1
FMNL2
FNIP1
FNIP2
FOLR1
FOS
FOSL1
FOXF2
FOXO1
FOXQ1
FRMD4A
FRMD5
FST
FZD8
GAS2L3
GDF15
GEM
GMPR
GNPDA1
GPCPD1
GPNMB
GPR146
GPRC5C
GPRIN3
GREB1
GRIP1
HCN3
HHAT
HIP1R
HMGA2
HMGCR
HMGCS1
HMMR
HMOX1
HRK
HS1BP3
HSD17B7
HSPB7
ICAM1
IDI1
IFI30
IFIT3
IGFN1
IL17RD
IL18R1
IL1R1
IL21R
IL24
IL6
IL6R
INPPL1
INSIG1
INTS6L
IRAK2
IRS2
ITGA7
ITPRIP
KCNJ2
KCNK2
KDM3A
KIF26A
KLC3
KLHL24
KLHL3
KLHL30
KLRG1
LAMA1
LAMA5-AS1
LANCL3
LBHD1
LDLR
LHFPL2
LIF
LINC00313
LINC01537
LINC01572
LINC01989
LIPG
LPAR5
LPIN1
LRP8
LRRC32
LSS
LYST
MAFB
MAFF
MALAT1
MBP
MCM3AP-AS1
MCOLN3
MEGF6
METTL7B
MGAT5B
MMAB
MORN4
MOSPD1
MSMO1
MT1E
MT1F
MT1G
MT1H
MT1M
MT1X
MT2A
MTHFR
MVD
MVK
MX1
MYLK3
MYO16
N4BP3
NCR3LG1
NEAT1
NEU1
NFIL3
NGF
NIPAL4
NOV
NPAS1
NPAS2
NPAS4
NPC1
NPIPA1
NPIPB11
NPIPB3
NPIPB4
NPIPB5
NSDHL
NUPR1
NYAP2
ORAI3
OSGIN1
OSMR-AS1
PADI1
PAG1
PANK3
PCDHB12
PCSK9
PCYT2
PDE2A
PDE5A
PDK1
PDK4
PDP1
PDP2
PER3
PFKFB2
PGF
PHF7
PHLDA1
PI4K2A
PIF1
PIM3
PKD1P6-NPIPP1
PLA2G3
PLA2G6
PLB1
PLCG2
PLCL1
PLEKHG1
PLEKHM1
PLS3-AS1
PLXNB3
PNPLA3
POR
PPARGC1A
PPM1H
PPP1R15A
PPP1R3B
PPP1R3G
PRAG1
PRKAG2
PRR15
PSCA
PSG4
PTGES
PTGS2
PTPRB
PTPRE
PTPRO
PTPRR
PTX3
PXDC1
RAB17
RAB27B
RAB29
RAB3IL1
RAB4B
RAP1GAP
RAPGEF3
RAPGEFL1
RASEF
RBM12B-AS1
RGS17
RGS2
RHEBL1
RILP
RIMS3
RIMS4
RNF125
RNF144A
RNF43
RORC
RRAGC
RRAGD
RSPO3
RTN4R
RTN4RL2
RUSC1-AS1
SAT2
SC5D
SCD
SCG2
SCML1
SERTM1
SGPP2
SH2D5
SH3TC2
SLC12A7
SLC16A13
SLC26A2
SLC2A3
SLC2A6
SLC36A1
SLC38A7
SLC9A2
SLC9A9
SLFN5
SMIM29
SMOX
SNAI3
SNAI3-AS1
SNN
SNX8
SPATAI 2
SPATA25
SPIRE1
SPRED1
SPRED2
SPRY2
SPRY4
SQLE
SQSTM1
STAG3L5P-
PVRIG2P-
PILRB
STARD4
STC2
STEAP1
STK33
STRIP2
STX11
STX3
SV2A
SYNE3
TACR1
TBC1D2
TCEANC
TDRKH
TESK2
THAP8
TLR4
TM7SF2
TMEM144
TMEM156
TMEM192
TMEM251
TMEM37
TMEM51
TMEM52
TMEM97
TNFAIP3
TNFAIP8L3
TNFRSF14
TNFSF9
TRAPPC6A
TRIB3
TRIM2
TSNARE1
TSPAN10
TTC9
U2AF1
UAP1L1
UBASH3B
ULBP1
UPP1
USP51
VASH2
VEGFA
VEGFC
VGLL4
VLDLR
VSIG10L
WDR31
WDSUB1
WIPI1
WSB1
ZBED6CL
ZCCHC14
ZDHHC11B
ZNF165
ZNF19
ZNF395

In some embodiments, a TRPML1 agonist is characterized in that TRPML1 level or activity is higher in its presence than in its absence, under comparable conditions. In some embodiments, a TRPML1 agonist is a direct agonist in that it interacts with TRPML1. In some embodiments, a TRPML1 agonist is an indirect agonist in that it does not directly interact with TRPML1.

In some embodiments, the present disclosure provides a method of activating TFEB by enhancing GABARAP/FNIP/FLCN complex localization at an intracellular membrane surface. In some embodiments, an intracellular membrane surface is a cytosolic surface of an intracellular compartment. In some embodiments, an intracellular compartment is a lysosome. In some embodiments, an intracellular compartment is a mitochondria. In some embodiments, an intracellular compartment is an endoplasmic reticulum. In some embodiments, an intracellular compartment is a pathogen vacuole. In some embodiments, an intracellular compartment is an endosomal structure. In some embodiments, the method comprises administering a TRPML1 agonist. In some embodiments, TFEB activation is independent of mTORC1 activity.

In some embodiments, the present disclosure provides active agents for use as reference or control for identification or characterization of other active agents. In some embodiments, the present disclosure provides a method of characterizing a TFEB activating agent, the method comprising assessing effect on FLCN localization and/or level of a GABARAP/FNIP/FLCN complex at one or more intracellular membrane surfaces.

In some embodiments, the present disclosure provides a method comprising a cellular assay for characterizing activators of TFEB, TFE3 and/or MITF, wherein the cellular assay comprises cells comprising presence of a vATPase small molecule inhibitor; genetic disruption of ATG8 conjugation machinery; presence of a small molecule inhibitor of ATG8 conjugation machinery; genetic disruption of a member of a GABARAP subfamily of proteins; mutation of a LIR domain in FNIP1 or FNIP2, or a combination thereof. In some embodiments, a vATPase small molecule inhibitor is Bafilomycin A1. In some embodiments, a vATPase small molecule inhibitor is not an analogue of Salicylihalamide A. In some embodiments, a genetic disruption of ATG8 conjugation machinery comprises knock-out of a gene, knock-in of a gene, expression of one or more mutant alleles, siRNA, shRNA, antisense, or a combination thereof. In some embodiments, a genetic disruption of the member of a GABARAP subfamily of proteins comprises knock-out of a gene, knock-in of a gene, expression of one or more mutant alleles, siRNA, shRNA, antisense, or a combination thereof.

### Diseases, Disorders or Conditions

In some embodiments, the present disclosure provides a method of treating a TRPML1-associated disease, disorder or condition, the method comprising a step of administering a TRPML1 agonist to a subject suffering from, or susceptible to, the TRPML1-associated disease, disorder or condition. In some embodiments, a TRPML1-associated disease, disorder or condition is or comprises an inflammatory condition. In some embodiments, a TRPML1-associated disease, disorder or condition is or comprises a lysosomal storage disorder. In some embodiments, a TRPML1-associated disease, disorder or condition is or comprises a polyglutamine disorder. In some embodiments, a TRPML1-associated disease, disorder or condition is or comprises a neurodegenerative proteinopathy. In some embodiments, a TRPML1-associated disease, disorder or condition is or comprises an infectious disease. In some embodiments, a TRPML1-associated disease, disorder or condition is selected from a group consisting of Crohn's Disease, Pompe Disease, Parkinson's Disease, Huntington's Disease, Alzheimer's Disease, Spinal-bulbar muscular atrophy, α-1-antitrypsin deficiency, and multiple sulfatase deficiency. In some embodiments, a TRPML1-associated disease, disorder or condition is Crohn's Disease.

In some embodiments, the present disclosure provides that active agents as described herein may be particularly useful in the treatment of one or more conjugation-machinery-associated (“CMA”) diseases, disorders or conditions. In some embodiments, the present disclosure provides a method of treating a conjugation-machinery-associated (“CMA”) disease, disorder or condition or a GABARAP/FNIP/FLCN complex-associated disease, disorder or condition, the method comprising a step of administering a TRPML1 agonist. In some embodiments, the disease, disorder or condition is or comprises Crohn's Disease.

In some embodiments, a CMA disease disorder or condition is one that has been established to be associated with a mutation in or allele of a conjugation machinery gene. For example, the T300A polymorphism in ATG16L1 is associated with an increased incidence of Crohn's disease. This polymorphism increases the likelihood of proteolytic processing of ATG16L1 to remove the C-terminal region extending from amino acid 300. The C-terminal region of ATG16L1 is important for conjugation of ATG8 family members to single membranes and known to be important for the host-pathogen response. In the intestine, decreased ATG16L1 C-terminal function contributes to the proinflammatory nature of Crohn's disease, potentially through a lack of ATG16L1-CTD domain dependent TFEB activation. In some embodiments, treatment of this condition with an active agent (e.g. TRPML1 agonist or other agent) as described herein to restore full TFEB activity through agonism of intact full length ATG16L1 as described herein could be beneficial.

Germline mutations in FLCN underlie the FLCN-FNIP complex loss-of-function phenotype and TFEB-dependency in Birt-Hogg-Dube syndrome (35, 44), a rare disorder that predisposes patients to kidney tumors. Additionally, Ras-driven pancreatic adenocarcinoma cells show constitutive nuclear localization of TFEB/TFE3 and notable co-localization of LC3 with LAMP2-positive lysosomes (45, 46). Understanding the involvement of FLCN-FNIP complex membrane sequestration and whether oncogenic signals take advantage of this mechanism to drive TFEB-dependent tumor growth may offer new therapeutic opportunities against lysosome-dependent tumors. Elevated lysosomal activity or membrane permeability (46) could trigger this pathway and explain the TFEB nuclear localization despite full nutrient, mTOR-active conditions (45). In cancers with constitutively nuclear TFEB/TFE3/MITF, specific disruption of the GABARAP-FNIP interaction may allow for inhibition of TFEB/TFE3/MITF activity in certain tumors and decrease tumor progression.

In some embodiments, a conjugation-machinery-associated (“CMA”) disease, disorder or condition or a GABARAP/FNIP/FLCN complex-associated disease, disorder or condition, is or comprises a cancer. In some embodiments, a cancer is characterized by having nuclear localization of TFEB/TFE3 transcription factors. In some embodiments, a cancer is characterized by the presence of damaged endosome or lysosome structures. In some embodiments, a cancer is characterized by the presence of ATG8 homologs conjugated to intracellular membranes (e.g., endosomes, lysosomes, autophagosomes, or mitochondria).

### EXEMPLIFICATION

The following examples are provided so as to describe to the skilled artisan how to make and use methods and compositions described herein, and are not intended to limit the scope of the present disclosure.

### Materials and Methods

### Antibodies

ATG16L1 (8089, human), Phospho-ATG14 S29 (92340), ATG14 (96752), Phospho-Beclin S30 (54101), FIP200 (12436), FLCN (3697), GABARAPL1 (26632), GABARAPL2 (14256), GAPDH (5174, 1:10000 for WB), DYKDDDDK tag (14793), HA tag (3724), myc tag (2278), LC3A/B (12741), LC3B (3868), LAMTOR1 (8975), LAMP1 (15665, 1:1000 for IF), NFAT1 (5861, 1:250 for IF), NPRL2 (37344), Phospho-S6K (9234), S6K (2708), Phospho-S6 S235/236 (4858, 1:3000 for WB), S6 (2217, 1:5000 for WB), TAX1BP1 (5105), TFEB (4240), TFEB (37785, 1:200 for IF), and Phospho-ULK S757 (14202) antibodies used in these studies were from Cell Signaling Technologies. An FNIP1 antibody (ab134969) was from Abcam. A TFE3 antibody (HPA023881) was from Millipore Sigma. A p62 antibody (GP62-C) was from Progen. A Galectin-3 antibody (sc-23938) was from Santa Cruz Biotechnology. A TFEB antibody (A303-673A, 1:200 for IF in murine cells) was from Bethyl Laboratories. All antibodies were used at a 1:1000 dilution for Western blotting unless otherwise noted.

Generation of Knockout Cell Lines with CRISPR/Cas9

HeLa or U2OS cells were made to stably express Cas9 through lentiviral transduction (vector Cat #SVC9-PS-Hygro, Cellecta). Knockout cell lines were generated as pooled populations following subsequent lentiviral transduction with gRNA sequences as indicated (vector Cat #SVCRU6UP-L, Cellecta). Pooled populations were selected for 3 days with puromycin (2 ug/ml, Life Technologies) and used for experiments 7-9 days post-transduction with gRNA. Clones were isolated for ATG16L1_KO to use for reconstitution experiments. gRNA sequences (5′ to 3′) are provided in Table 2.

TABLE 2

| | |
|---|---|
| sgCTRL | GTAGCGAACGTGTCCGGCGT (SEQ ID NO: 1) |
| sgRB1CC1 | CAGGTGCTGGTGGTCAATGG (SEQ ID NO: 2) |
| (FIP200) | |
| sgATG9A | TCTGGAAACGGAGGATGCGG (SEQ ID NO: 3) |
| sgPIK3C3 | CATACACATCCCATATGGTG (SEQ ID NO: 4) |
| (VPS34) | |
| sgATG5 | GCTTCAATTGCATCCTTAGA (SEQ ID NO: 5) |
| sgATG7 | TCCGTGACCGTACCATGCAG (SEQ ID NO: 6) |
| sgATG16L1 | GCCACATCTCGGAGCAACTG (SEQ ID NO: 7) |
| sgLAMTOR1 | GCTGCTGTAGCAGCACCCCA (SEQ ID NO: 8) |
| sgNPRL2 | GATGCGGCAGCCGCTGCCCA (SEQ ID NO: 9) |
| sgMAP1LC3A | GTCAAGCAGCGGCGGAGCTT (SEQ ID NO: 10) |
| (LC3A) | |
| sgMAP1LC3B | GCAGCATCCAACCAAAATCC (SEQ ID NO: 11) |
| (LC3B) | |
| sgMAP1LC3C | GCTTGAAGGGTCTGACGCTT (SEQ ID NO: 12) |
| (LC3C) | |
| sgGABARAP | GGATCTTCTCGCCCTCAGAG (SEQ ID NO: 13) |
| sgGABARAPL1 | CATGAAGTTCCAGTACAAGG (SEQ ID NO: 14) |
| sgGABARAPL2 | TTCCCGCCGCCGCCATGAAG (SEQ ID NO: 15) |
| sgFLCN | TCACGCCATTCCTACACCAG (SEQ ID NO: 16) |
| sgFNIP1 | CAACATGTCCACACTCTGAG (SEQ ID NO: 17) |
| sgFNIP2 | CCAGTTGATATGCCAAGCAG (SEQ ID NO: 18) |

cDNA Expression Constructs

Wild type and K490A mutant mouse ATG16L1 were cloned into pBabe-Puro-Flag-S-tag plasmids as previously described (Fletcher et al EMBO J 2018). pBabe-Blast-GFP-LC3A has been previously described (Florey et al NCB 2011). The constructs listed in Table 3 were generated for use in these studies.

TABLE 3

| | Epitope | | Expression |
|---|---|---|---|
| Insert | Tag | Terminus | vector |
| Human FNIP1 WT | 3X HA | N | pCDNA-DEST40 |
| | | | Tet Lenti |
| Human FNIP1 | 3X HA | N | pCDNA-DEST40 |
| Y583A/V586A | | | Tet Lenti |
| Human FLCN WT | FLAG | C | pCDNA-DEST40 |
| | | | Tet Lenti |
| Human lyso-FLCN | FLAG | C | Tet Lenti |
| (N-terminus 39aa | | | |
| of LAMTOR1 fusion) | | | |
| Human GABARAP WT | myc | N | pcDNA-DEST40 |
| Human GABARAP LBMmut | myc | N | pcDNA-DEST40 |
| Human ATG16L1 WT | FLAG | N | Tet Lenti |
| Human ATG16L1 ΔCTD | FLAG | N | Tet Lenti |
| Human ATG16L1 ΔFBD | FLAG | N | Tet Lenti |
| Human ATG16L1 F467A | FLAG | N | Tet Lenti |
| Human ATG16L1 K490A | FLAG | N | Tet Lenti |
| S. Typhimurium SopF | myc | N | Tet-Lenti |
| Human LAMP1 | RFP | C | pBabe |

cDNA constructs with the indicated epitope tags were synthesized (Genscript, USA) and provided as entry clones. Gateway recombination was used to shuttle cassettes into pcDNA-DEST40 (Life Technologies) or a lentiviral vector, allowing for tetracycline inducible expression referred to as Tet-Lenti (synthesized by Genscript, USA).

### Cell Culture

The studies described below used U2OS, HeLa, and RAW264.7 cell lines, which were obtained from the American Type Culture Collection (ATCC). HEK293FT cells were obtained from ThermoFisher Scientific. Cell lines were verified to be mycoplasma-free by routine testing. All cells were cultured in a humidified incubator at 37° C. and 5% CO₂. Cell culture reagents were obtained from Invitrogen unless otherwise specified. Cells were grown in Dulbecco's Modified Eagle's Medium (DMEM) supplemented with 10% fetal bovine serum and 1% penicillin/streptomycin. RAW264.7 wild type and ATG16L1_KO cells were provided by Dr. Anne Simonsen (Lystad et al NCB) and maintained in DMEM 10% FBS, 1% pen/strep.

### Reagents

Bafilomycin A1, PIK-III, and AZD8055 were purchased from Selleckchem. ML-SA1 and MK6-83 were purchased from Tocris. Monensin, nigericin, salinomycin, valinomycin, and LLoMe were purchased from Sigma Aldrich. C8 is available for purchase through Chemshuttle (Cat #187417).

### Viral Production and Transduction

For lentiviral production of CRISPR gRNA or Cas9 virus and cDNA overexpression virus, 8×10⁵ 293FT cells were plated in 6-well plates. The next day, cells were transfected with lentiviral packaging mix (1 μg psPAX2 and 0.25 μg VSV-G) along with 1.5 μg of the lentiviral backbone using Lipofectamine 2000 (ThermoFisher). Supernatant was removed from 293FT cells after 48 hours, centrifuged at 2000 rpm for 5 minutes and then syringe filtered using a 0.45 μm filter (Millipore). Polybrene was then added to a final concentration of 8 μg/ml and target cells were infected overnight. Cells were then allowed to recover for 24 hours in DMEM/10% FBS before being selected with 1 mg/mL neomycin (G418:Geneticin, ThermoFisher), 2 μg/mL puromycin (ThermoFisher), or 500 μg/mL Hygromycin B (ThermoFisher) for 72 hours.

Retroviral infection was performed as described previously (Gammoh et al NSMB 2013) using centrifugation. Stable populations were selected with puromycin (2 mg/mL) or blasticidin (10 mg/mL) for 3-5 days.

### Cell Lysis and Western Blotting

For preparation of total cell lysates, cells were lysed in RIPA buffer (#9806, Cell Signaling Technology) supplemented with sodium dodecyl sulfate to 1% final concentration (SDS, Boston BioProducts) and protease inhibitor tablets (Complete EDTA-free, Roche). Lysates were homogenized by sequential passaging through Qiashredder columns (Qiagen), and protein levels were quantified by Lowry DC protein assay (Bio-Rad). Proteins were denatured in 6×Laemmli SDS loading buffer (Boston BioProducts) at 100° C. for 5 minutes.

1.5×10⁶ cells were plated the day before in 6 cm tissue culture dishes (BD Falcon) for preparation of membrane fractions. Cellular fractions were prepared using the MEM-PER kit (ThermoFisher) according to the manufacturer's protocol. Protein levels were quantified by Lowry DC protein assay (Bio-Rad) and denatured in 6×Laemmli SDS loading buffer (Boston BioProducts).

Equivalent amounts of total proteins were separated on Tris-Glycine TGx SDS-PAGE gels (Bio-Rad) for Western blotting. Proteins were transferred to nitrocellulose using standard methods and membranes were blocked in 5% non-fat dry milk (Cell Signaling Technology) in TBS with 0.2% Tween-20 (Boston BioProducts). Primary antibodies were diluted in 5% bovine serum albumin (BSA, Cell Signaling Technology) in TBS with 0.2% Tween-20 and were incubated with membranes at 4° C. overnight. HRP-conjugated secondary antibodies were diluted in blocking solution (1:20,000, ThermoFisher) and incubated with membranes at room temperature for 1 hour. Western blots were developed using West PicoPLUS Super Signal ECL reagents (Pierce) and film (GEHealthcare).

### Immunoprecipitation

Cells were lysed in IP CHAPS lysis buffer: 0.3% CHAPS, 10 mM O-glycerol phosphate, 10 mM pyrophosphate, 40 mM Hepes pH 7.4, 2.5 mM MgCl₂, supplemented with protease inhibitor tablets (Roche) and Calyculin A (Cell Signaling Technology). Lysates were clarified by centrifugation and equilibrated as described above. For FLAG IP, lysates were incubated with anti-M2 FLAG-conjugated agarose beads (Sigma-Aldrich) at 10 μL bed volume per 1 mg of protein and incubated for 1 hour at 4° C. with gentle rocking. For MYC IP, lysates were incubated with 10 μL bed volume per 1 mg protein of anti-myc 9E10-conjugated agarose beads (Sigma Aldrich). Beads were then centrifuged and washed with lysis buffer 3 times. Immunoprecipitate was eluted by addition of 6×Laemmli SDS loading buffer at 100° C. for 5 minutes.

### Immunofluorescence and High Content Image Analysis

Following indicated treatments, GFP-LC3 LAMP1-RFP expressing cells were fixed with ice cold methanol for 3 minutes at −20° C. Cells were washed in PBS and image acquisition was performed using a Confocal Zeiss LSM 780 microscope (Carl Zeiss Ltd) equipped with a 40×oil immersion 1.40 numerical aperture (NA) objective using Zen software (Carl Zeiss Ltd).

For quantification, the number of GFP-LC3 puncta were counted for >20 cells across multiple fields of view. For co-localization quantification, GFP-LC3 puncta were assessed for LAMP1-RFP.

For LC3 and LAMP1 staining in primary BMDMs, cells were plated on 18 mm coverslips. The next day, cells were treated as indicated and cells were fixed in ice cold methanol as described above. Cells were then blocked in PBS+5% BSA for 1 hour before addition of primary antibodies (anti-LC3A/B, CST #4108, 1:100; anti-LAMP1, BD #555798, 1:100) and diluted in blocking buffer overnight at 4° C. Cells were then washed and incubated with fluorescent secondary antibodies in blocking buffer for 1 hour at room temperature. Cells were washed in PBS incubated with DAPI and mounted on glass slides using Prolong anti-fade reagent (Life Technologies).

For endogenous TFEB staining in mouse macrophages, cells were fixed in 3.7% formaldehyde for 15 minutes at room temperature, washed in PBS and permeabilized in 0.2% triton/PBS for 5 minutes. Cells were then processed as above for primary (anti-TFEB, Bethyl Laboratories, #A303-673A, 1:200) and secondary antibodies. Images were acquired using a Confocal Zeiss LSM 780 microscope (Carl Zeiss Ltd) equipped with a 40×oil immersion 1.40 numerical aperture (NA) objective using Zen software (Carl Zeiss Ltd). Analysis was performed using Image J. For nuclear cytosol quantification, the ratio of fluorescent intensity of TFEB within the DAPI was mask versus the cytosol of 30 cells across 2 independent experiments were measured.

For high content image acquisition, cells were plated in 96-well glass bottom, black wall plates (Greiner, #655892) or 384-well polystyrene, black-wall plates (Greiner, #781091) and grown overnight to 70% confluency. Treatments were performed as indicated. Cells were fixed for 10 minutes in either −20° C. methanol or 4% paraformaldehyde (Electron Microscopy Sciences). Cells were blocked and permeabilized in a solution containing 1:1 Odyssey blocking buffer (LiCor)/PBS (Invitrogen) with 0.1% Triton X-100 (Sigma) and 1% normal goat serum (Invitrogen) for 1 hour at room temperature. Primary antibodies were added overnight at 4° C. in blocking buffer described above. After washing plates with PBS using an EL-406 plate washer (BioTek), secondary Alexa-conjugated antibodies (Life Technologies) were diluted 1:1,000 in blocking solution and applied for 1 hour at room temperature. Cells were then washed again in PBS as described and imaged using an INCELL 6500 high-content imager (GE Healthcare). Images were analyzed using the GE InCarta software.

### Correlative FIB-SEM

Cells were seeded in 35 mm glass-bottom dishes (MatTek Corp., USA, #P35G-2-14-CGRD). They were fixed with 4% formaldehyde (TAAB F017, 16% w/v solution formaldehyde-methanol free) in 0.1 M phosphate buffer pH 7.4 (PB) for 30 minutes at 4° C. They were washed in PB and imaged with a 40×/1.4 NA objective on an inverted confocal microscope (Zeiss LSM780). Further fixation was carried out with 2% formaldehyde and 2.5% glutaraldehyde (TAAB G011/2, 25% solution glutaraldehyde) in PB for 2 hours, prior to further processing.

Samples were embedded using a protocol as described previously (38, 39). The cells were washed in PB five times and post-fixed in 1% osmium tetroxide (Agar Scientific, R1023, 4% solution osmium tetroxide) and 1.5% potassium ferrocyanide (v/v) (SIGMA ALDRICH, P3289-100G, potassium hexacyanoferrate (II) trihydrate) for 1 hour on ice. Samples were then dehydrated and embedded in Hard-Plus Resin812 (EMS, #14115). The samples were polymerized for 72 hours at 60° C. The coverslip was removed from the resin by dipping the block into liquid nitrogen. After locating the region of interest (ROI) on the block surface using the imprint of the grid, the block was cut to fit on an aluminium stub using a hacksaw, and trimmed with a razorblade. The block/stub was then coated with 20 nm Pt using a Safematic CCU-010 sputter coater (Labtech) to create a conductive surface.

The block/stub was placed in the chamber of a Zeiss 550 CrossBeam FIB SEM and the surface imaged using the electron beam at 10 kV to locate the grid and underlying cells. Once the ROI had been identified, Atlas software (Fibics) was used to operate the system. A trench was cut into the resin to expose the target cell and serial SEM images were acquired with 7 nm isotropic resolution using a 1.5 kV electron beam. For 3D-image analysis, image stacks were processed using Atlas software and viewed using ImageJ.

### RNA Isolation and RNAseg Analysis

RNA Isolation

Total RNA was prepared from cells treated with DMSO or 2 μM C8 for 24 hours using Trizol extraction and RNAeasy Mini Kit (Qiagen). A total of 2 μg of RNA with a RIN score of >9.8 was submitted for RNAseq analysis.

Library Preparation, HiSeq Sequencing and Analysis

RNA sequencing libraries were prepared using the NEBNext Ultra RNA Library Prep Kit for Illumina using manufacturer's instructions (NEB, MA). mRNAs were enriched with Oligod(T) beads, and the enriched mRNAs fragmented at 94° C. for 15 minutes. This was followed by first strand and second strand cDNA synthesis cDNA fragments were end-repaired and adenylated at 3′ends. Universal adapters were then ligated to cDNA fragments, followed by index addition and library enrichment by PCR with limited cycles. The sequencing library and RNA samples for RNAseq were quantified using Qubit 2.0 Fluorometer (Life Technologies, CA) and RNA integrity checked using Agilent TapeStation 4200 (Agilent Technologies, CA).

The sequencing libraries were clustered on a single lane of a flowcell on the Illumina HiSeq 4000 system according to manufacturer's instructions. The samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification. Sequence reads were mapped to the Homo sapiens reference genome version GRCh38 available on ENSEMBL using the STAR aligner v.2.5.2b. Unique gene hit counts were calculated by using feature Counts from the Subread package v.1.5.2. Only unique reads that fell within exon regions were counted.

The count data was normalized by the trimmed mean of M-values normalization (TMM) method, followed by variance estimation and applying generalized linear models (GLMs), utilizing functions from empirical analysis of digital gene expression (40) to identify differentially expressed genes as described previously (41, 42). Factorial designs were incorporated into the analysis by fitting these linear models with the coefficient for each of the factor combinations and then simultaneously extracting contrasts for the respective ‘differential-of-differential’ analysis in the two experimental dimensions (C8 stimulation and genotype status: ATG16L1KO and WT). The associated p-values were adjusted to control the false discovery rate in multiple testing, using the Benjamini and Hochberg's (BH) method (BH-adjusted p<0.05).

Pathway and biological process enrichment analysis were performed as previously described (42, 43). Briefly, data were interrogated from KEGG pathways and gene ontology biological processes. Each module or category was assessed for statistical enrichment or over-representation among differentially expressed genes relative to their representation in the global set of genes in the genome. P-values were computed using the hypergeometric test.

Lysotracker Staining

U2OS.Cas9 cells expressing a control gRNA or knocked out for ATG16L1 were treated for 24 hours with 2 μM C8. Cells were then washed and incubated live for 20 minutes with 25 nM Lysotracker Red DND-99 (ThermoFisher) and Hoecsht 33342 (ThermoFisher) diluted in warmed imaging buffer (20 mM HEPES (pH 7.4), 140 mM NaCl, 2.5 mM KCl, 1.8 mM CaCl2, 1 mM MgCl2, 10 mM D-glucose, and 5% v/v FBS). Staining solution was removed and cells were incubated in imaging buffer for an additional 30 minutes before image acquisition on the INCELL 6500. Images were analyzed using the GE InCarta software.

Generation of ATG16L1 K490A Knockin Mouse Model

The K490A point mutation was introduced into C57/BL6 mice via direct zygote injection of CRISPR/Cas9 reagents. Briefly, a single stranded guide sequence was designed and synthesized along with a tracrRNA from Dhamacon. A repair donor single stranded DNA sequence was designed to introduce the K490A point mutation and mutate the PAM sequence to stop re-targeting of the Cas9 complex to already edited DNA. These reagents, along with recombinant Cas9, were injected into mouse zygotes. Pups born from these injections were genotyped via Transnetyx and heterozogous founders were bred with wild-type mice to obtain pure heterozygote animals. Further breeding yielded mice homozygous for the K490A mutation. Mice were housed in the Biological Support Unit at the Babraham Institute under specific pathogen-free conditions.

K490A guide sequence:
(SEQ ID NO: 19)
GUUAGGGGCCAUCACGGCUCGUUUUAGAGCUAUGCUGUUUUG
Repair donor ssDNA:
(SEQ ID NO: 20)
GCTGTCTCCCTTAGGTCAGAGAGAGTGTGGTCCGAGAGATGGAACTGTT
AGGGGCGATCACCGCTTTGGACCTAAACCCTGAGAGAACTGAGCTCCTG
AGCTGCTCCCGTGATGACCTG

Bone Marrow Derived Macrophage Isolation

C57/BL6 wild-type and ATG16L1 K490A mice, aged 13-15 weeks, were used to obtain bone marrow derived cells (BMDCs). Bone marrow cells were isolated by flushing tibias and femurs with PBS+2% FBS. Cells were pelleted and resuspended in 1 mL Red Blood Cell lysis buffer (150 mM NH₄Cl, 10 mM KHCO₃, 0.1 mM EDTA) for 2 minutes at room temperature. Cells were pelleted and resuspended in RPMI 1640 (Invitrogen 22409-031), 10% FBS, 1% Pen/Strep, 50 μM 2-mercaptoethanol supplemented with 20 ng/ml M-CSF (Peprotech #AF-315-02) and 50 ng/mL Fungizone (Amphotericin B) (Gibco #15290018). Media was refreshed on days 3 and 6 and cells plated for assays on day 8.

HEK293 GFP-LC3B ATG13/ATG16L1_DKO Cells

ATG13_KO HEK293 cells stably expressing GFP-LC3B maintained in DMEM, 10% FBS, 1% pen/strep, were used as previously described (Jacquin et al Autophagy 2017). To generate ATG16L1 KO, a gRNA sequence (GTGGATACTCATCCTGGTTC (SEQ ID NO: 21)) with overhangs for containing a BpiI site was annealed and cloned into the pSpCas9(BB)-2A-GFP plasmid (Addgene, 48138; deposited by Dr. Feng Zhang) digested with the BpiI restriction enzyme (Thermo Scientific, ER1011). The recombinant plasmid along with a pBabe-puro construct (Addgene, 1764; deposited by Dr. Hartmut Land) expressing mouse ATG16L1 variants was transfected into HEK293 ATG13_KO GFP-LC3B cells via Lipofectamine 2000 (Invitrogen). Cells were selected with 2.5 μg/ml puromycin (P8833, Sigma) for 48 hours, and single cell clones were obtained by limiting dilution. After clonal expansion, ATG16L1 KO clones were selected based on the absence of ATG16L1 protein as detected by Western blot.

Live Imaging Time-Lapse Confocal Microscopy

HEK293 cells were plated on 35 mm glass-bottomed dishes (Mattek, Ashland, Mass.). Images were acquired every 2 minutes using a spinning disk confocal microscope, comprising Nikon Ti-E stand, Nikon 60×1.45 NA oil immersion lens, Yokogawa CSU-X scanhead, Andor iXon 897 EM-CCD camera and Andor laser combiner. All imaging with live cells was performed within incubation chambers at 37° C. and 5% CO₂. Image acquisition and analysis was performed with Andor iQ3 (Andor Technology, UK) and ImageJ.

Endogenous Calcium Imaging

Hela wild-type and Hela ATG16L1KO cells were trypsinized and seeded at 20000 per well of PDL coated Greiner Bio plates for 2 hours. Cells were loaded with 10 μL of Calcium 6 dye solution for 1.5 hours at room temperature. After incubation, the dye was removed from the plates and replaced with 10 μL of low Ca²⁺ solution containing 145 mM NaCl, 5 mM KCl, 3 mM MgCl2, 10 mM glucose, 1 mM EGTA and 20 mM HEPES at pH 7.4. With 1 mM EGTA, the free Ca²⁺ concentration is estimated to be <10 nM based on the Maxchelator software. Compounds plates were prepared with low calcium solution. Cell and compound plates were loaded onto the FLIPR and a 15 minute protocol was run. The fluorescence intensity at 470 nm was monitored. After an initial 10 second baseline read, compounds were added to the cells. Images were taken for 15 minutes to monitor effects on Ca²⁺ fluorescence. Data exported as max-min Relative Fluorescence Unit (RFU).

### Recombinant Protein Expression

Purification of FLCN/FINP2 and GABARAP_MBP

Full-length human FNIP2 and FLCN were subcloned and purified as described in [21]. Final purified complexes were snap frozen in liquid nitrogen in buffer A (25 mM HEPES pH 7.4, 130 mM NaCl, 2.5 mM MgCl₂, 2 mM EGTA, and 0.5 mM TCEP). Full-length human GABARAP (1-117) was subcloned with a C-terminal MBP tag (GABARAP_MBP) separated by a GSSGSS linker in pET21b and expressed in E. coli following induction at 16° C. for 16 hours in LB. Cells were lysed in 50 mM Tris pH 7.4, 500 mM NaCl, 0.5 mM TCEP, 0.1% Triton X-100, 1 mM PMSF, and 15 μg/mL benzamidine; sonicated; and clarified by centrifugation. GABARAP_MBP was purified using amylose resin equilibrated in wash buffer (50 mM Tris pH 7.4, 500 mM NaCl, 0.5 mM TCEP) and eluted with wash buffer plus 30 mM maltose. The protein was further purified by size exclusion chromatography using a Superdex 75 column equilibrated buffer A and snap frozen in liquid nitrogen.

Purification of FLCN/FNIP2/GABARAP_MBP Complex

Purified GABARAP_MBP was mixed with FLCN/FNIP2 at a ratio of 1:0.8 and gently mixed for 2 hours at 4° C. The sample was injected onto a Superose 6 Increase (GE) column (1CV=24 mL) that was pre-equilibrated in Buffer A. The retention time of peak fractions were compared to FLCN/FNIP2 and GABARAP_MBP alone followed by evaluation of samples using 12% SDS-PAGE.

In Vitro FLCN-FNIP-GABARAP Complex Analysis

Purified GABARAP_MBP was complexed with FLCN/FNIP2 at a ratio of 1:0.8 and gently mixed for 2 hours at 4° C. The sample was then injected onto a Superose 6 Increase (GE) column (1CV=24 mL) that had been pre-equilibrated with Buffer A (25 mM HEPES, 130 mM NaCl, 2.5 mM MgCl₂, 2 mM EGTA, 0.5 mM TCEP, pH 7.4). Fractions (0.5 mL) were collected and the samples were analyzed on a 12% SDS-PAGE.

GEE Chemical Footprinting

Examination of a GABARAP-MBP interaction with FLCN/FNIP2 using GEE labeling chemistry and mass spectrometry techniques. The GABARAP-MBP and FLCN/FNIP2 protein samples were buffer exchanged against 1×PBS, 2.5 mM MgCl2, 0.5 mM TCEP buffer, pH 7.8. To form a GABARAP-MBP:FLCN/FNIP2 protein complex, 7.5 μM of GABARAP-MBP and 1.25 μM of FLCN/FNIP2 were mixed to maintain a molar ratio of GABARAP-MBP to FLCN/FNIP2 at 6:1. The protein concentration of the free FLCN/FNIP2 was adjusted to 1.25 μM. All samples were labeled by GEE for 0, 2.5, 5 and 6.5 minutes and subsequently precipitated with 10% TCA/acetone to clean up proteins. Then samples were reduced and alkylated with 10 mM iodoacetamide (IAM) and 25 mM of DTT, respectively, and digested with trypsin for overnight at 37° C. followed by digestion with Asp-N for 8 hrs at 37° C. Both trypsin and Asp-N were added to the protein samples at a 1:10 (w:w) enzyme:protein ratio. Next, digested samples were analyzed by liquid chromatography (nano-ACQUITY) coupled with high-resolution mass spectrometry (Eclipse). The MS data were analyzed by Mass Matrix software and manually, resulting in dose-response plots for each peptide. Results from the free FLCN/FNIP2 were compared against the GABARAP-MBP-FLCN/FNIP2 complex form. The standard approach for these studies is to assess overall decrease in labeling (protections) upon complex formation at the peptide level, and for peptides that have significant protections overall examine the modification of each residue within these peptides for its individual protections.

Ion homeostasis and acidic pH are tightly coupled determinants of lysosomal degradative capacity and alterations in these properties can cause disease. Although lysosomal membrane channels are known to regulate ion flux, how such changes are sensed locally by the lysosome and how the cell responds is unclear. The present Examples demonstrate that specific pharmacological alteration of lysosomal ion balance leads to vATPase-dependent recruitment of the ATG5-ATG12-ATG16L1 autophagy conjugation machinery to the lysosomal surface. ATPase-independent recruitment of the C-terminal WD40 domain of ATG16L1 enables direct conjugation of ATG8 homologs to the lysosomal membrane in an autophagy-independent manner. Importantly, conjugated GABARAP, but not LC3 proteins, sequester the FLCN/FNIP tumor suppressor complex to lysosomes. FLCN/FNIP sequestration abrogates regulation of RagC/D nucleotide state, resulting in nuclear accumulation of the transcription factor EB (TFEB) in an mTOR-independent manner. This novel ATG16L1-GABARAP-FLCN-TFEB axis facilitates lysosomal biogenesis in response to disruption in ion balance within the lysosomal network.

### Example 1

The present Example demonstrates that activation of the lysosomal ion channel TRPML1 results in ATG8 conjugation to the lysosomal membrane, independent of autophagy. ATG8 homologs (of the LC3 and GABARAP subfamilies) are widely used as markers of autophagosomes, double membrane bound structures that mediate the delivery of cytosolic contents to the lysosome for degradation and recycling (1). However, ATG8 proteins can also be conjugated to single-membrane organelles within the endocytic system, but the functional consequence of this modification is not well understood (2-6). Single-membrane ATG8 conjugation (SMAC), can be induced by pharmacological agents that exhibit lysosomotropic and ionophore/protonophore-like properties but that lack a molecular target (7, 8). To interrogate whether alterations in endolysosomal ion gradients serve as a trigger for ATG8 conjugation, pharmacological agonists of the lysosomal transient receptor potential mucolipin channel 1 (TRPML1) were used to acutely alter lumenal ion concentration. In the process, a novel mechanism that is responsible for maintaining organellar homeostasis was uncovered.

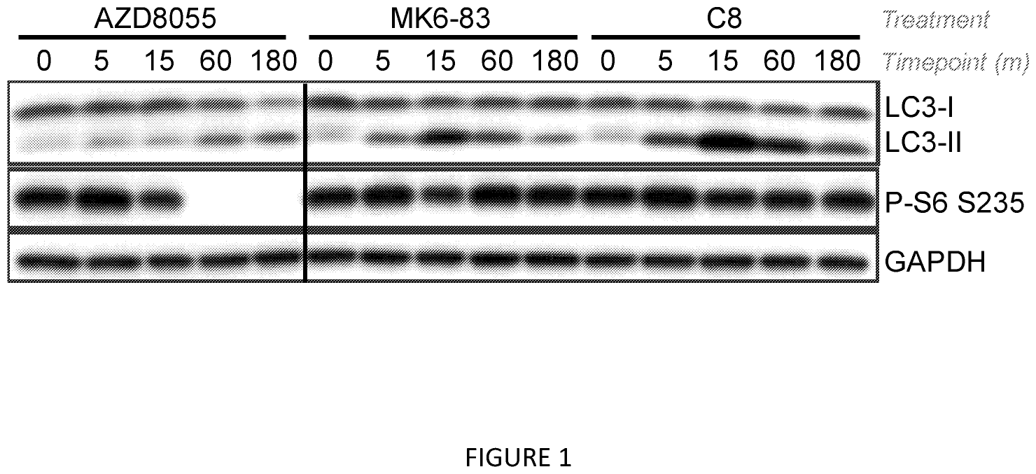

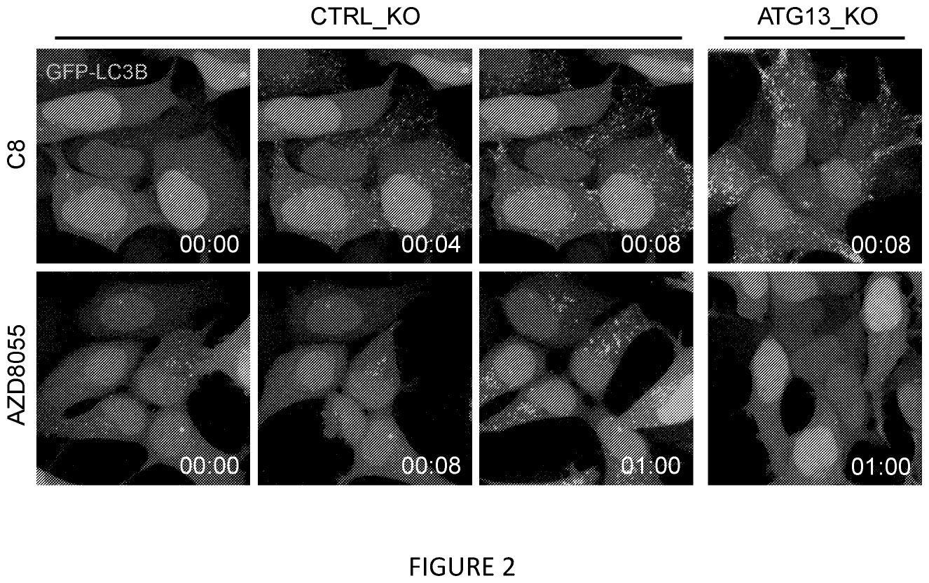

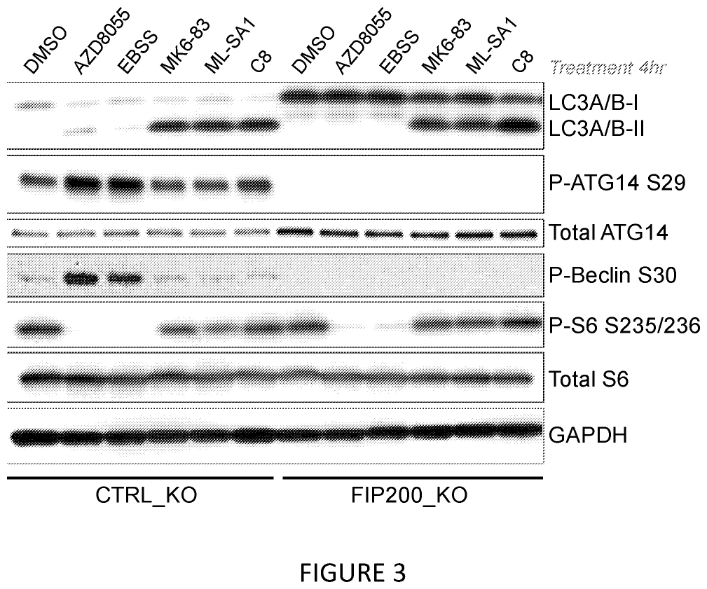

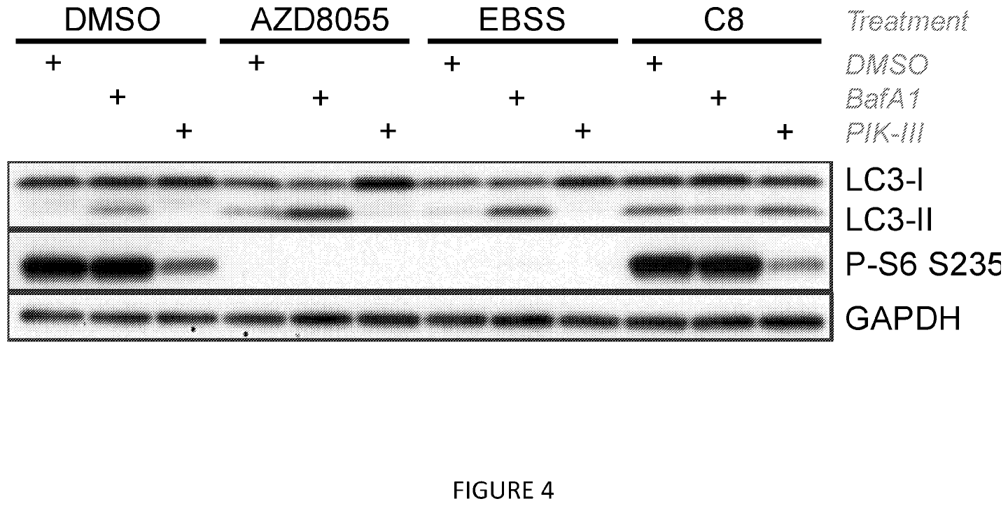

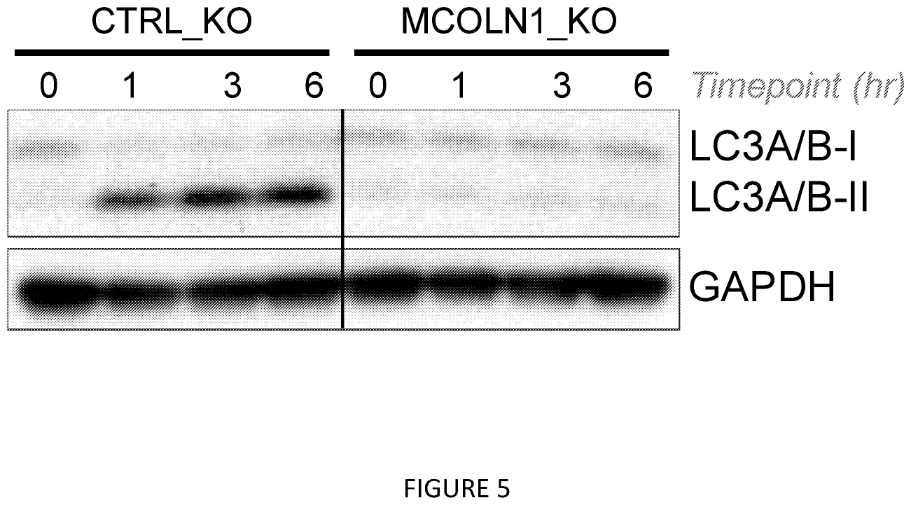

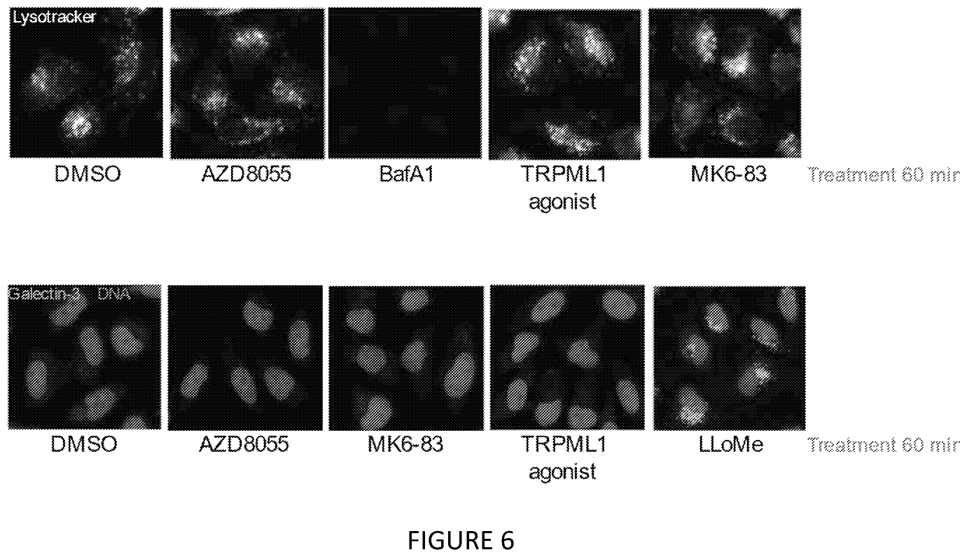

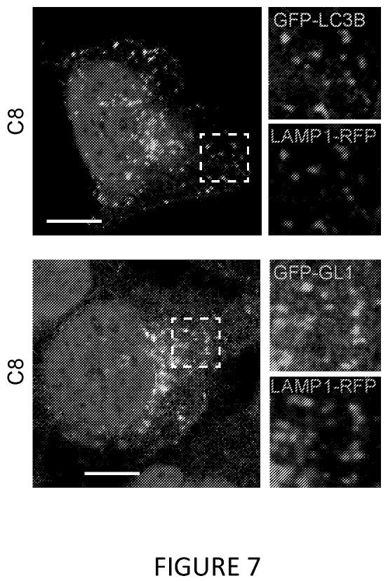

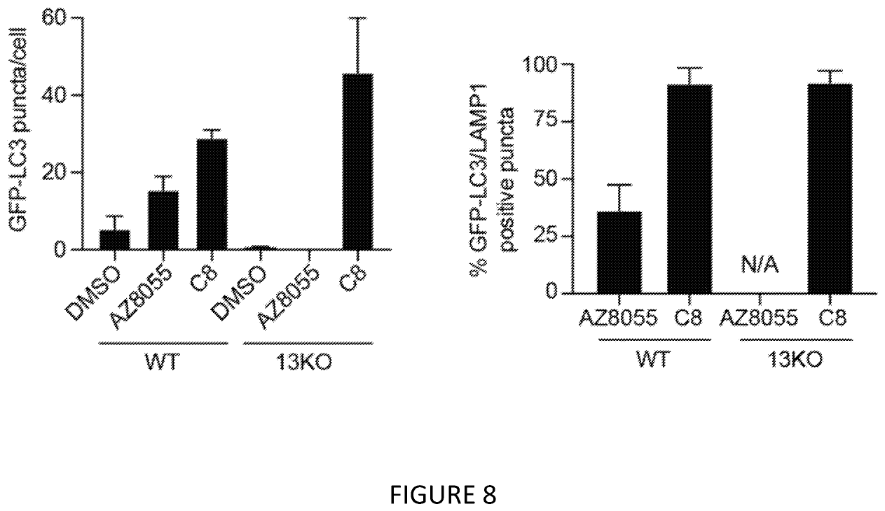

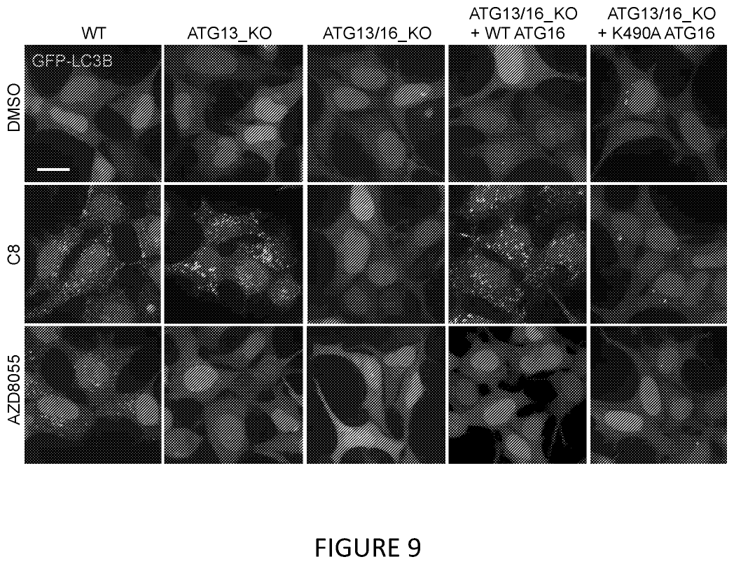

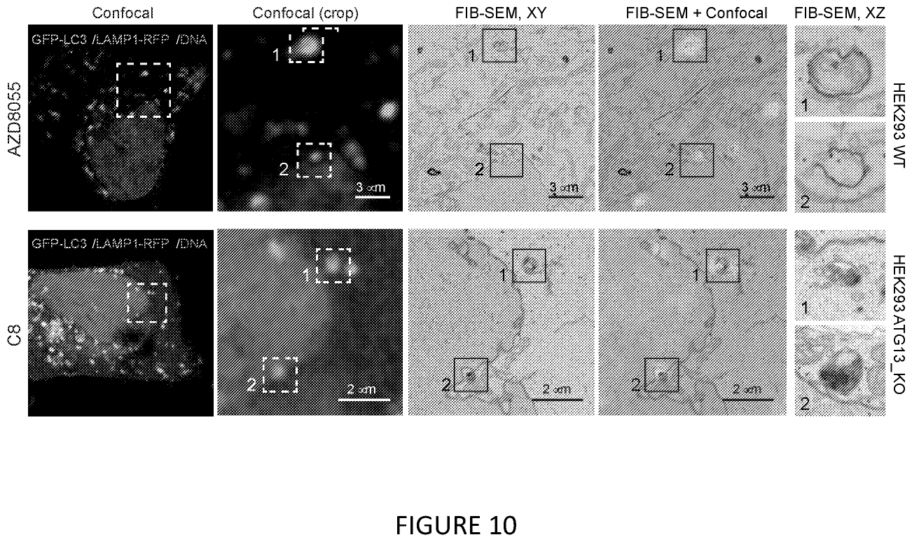

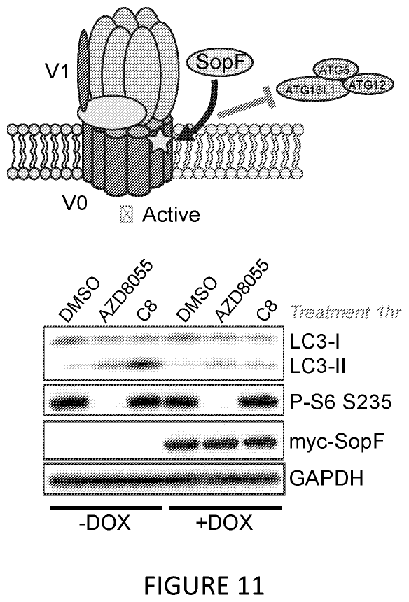

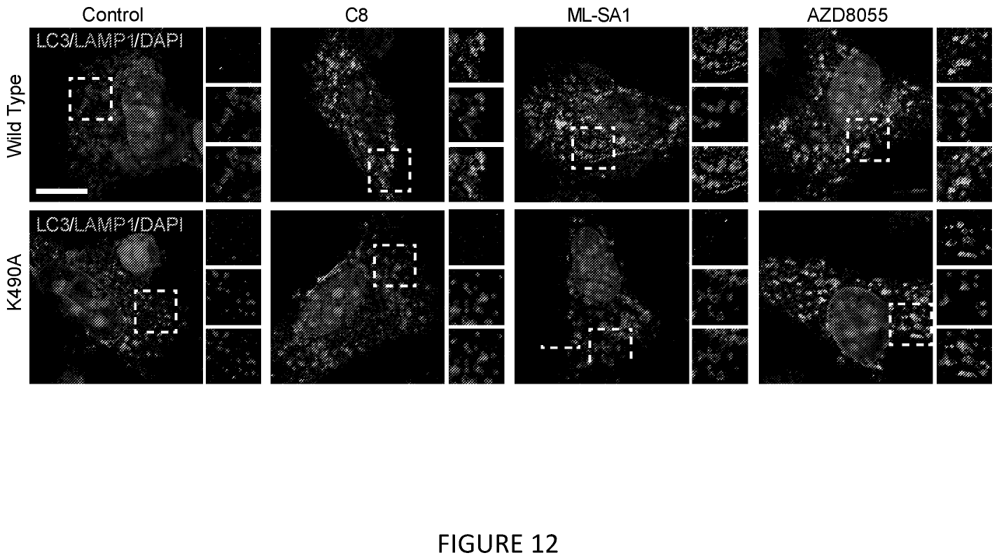

Treatment with the TRPML1 agonists MK6-83(9), ML-SA1(10) or a recently published, more potent channel agonist (designated as compound 8 “C8”)(11) resulted in the rapid conversion of LC3 from its cytoplasmic “I” form to the lipidated, punctate “II” form in both wild type (WT) and autophagy-deficient cells. In contrast, the mTOR inhibitor AZD8055, a well-established agent to induce autophagosome biogenesis, was able to regulate LC3 lipidation in WT but not autophagy-deficient cells (12) (FIGS. 1, 2, and 3). AZD8055 or EBSS starvation induced conversion of LC3 in a manner sensitive to VPS34 inhibition and was potentiated with the vATPase inhibitor Bafilomycin A1 (BafA1) (FIG. 4). Interestingly, treatment with C8 robustly induced VPS34-independent LC3 lipidation that was inhibited by BafA with no impact on mTOR activity (FIG. 4). This rapid lipidation depended on TRPML1 (FIG. 5) and was not accompanied by lysosomal alkalization or membrane damage (FIG. 6). Together, these features are characteristic of SMAC, where ATG8s are conjugated to endolysosomal membranes (7). Consistent with this, TRPML1 agonist treatment also induced strong co-localization of ATG8s (LC3B or GABARAPL1) with the lysosomal marker LAMP1 (FIGS. 7 and 8). Recent discoveries indicate that ATG8 conjugation to non-autophagosomal membranes requires distinct residues, such as K490, within the C-terminal WD repeats of ATG16L1, which are not required for autophagosome formation (13). Autophagy-deficient (ATG13 KO) cells were engineered to isolate the function of this ATG16L1 domain and it was found that TRPML1 agonist (e.g., C8)-induced LC3 puncta formation was abolished upon introduction of the ATG16L1 mutant K490A in ATG16L1 KO cells (FIG. 9). Further ultrastructural correlative light electron microscopy (CLEM) revealed GFP-LC3 structures characteristic of lysosomes (FIG. 10). It was recently reported that ATG16L1 recruitment to the Salmonella-containing vacuolar membrane required an interaction between the vATPase VOC subunit and ATG16L1 (8), and this recruitment was blocked by the bacterial effector protein SopF. FIG. 11 shows a diagram of Salmonella SopF impairment of ATG16L1 recruitment by the vATPase. Induction of SopF was sufficient to block LC3-II formation upon treatment with a TRPML1 agonist (C8), but not upon treatment with the canonical autophagy inducer (and mTOR inhibitor) AZD8055 (FIG. 11). Next, as shown in FIG. 12, a knock-in mouse model of the ATG16L1 K490A mutation was generated to specifically disrupt single membrane ATG8 conjugation. Mice were viable and did not exhibit any overt phenotypes, consistent with characterization of a mouse lacking the entire C-terminal domain of ATG16L1 (16). Primary bone marrow-derived macrophages were isolated from these animals and, as shown in FIG. 12, the LC3 puncta formation that co-localized with LAMP1 in wild-type cells after treatment with either of the TRPML1 agonists C8 and ML-SA1 did not occur in ATG16L1^{K490A} cells. This sensitivity to the ATG16L1^{K490A} mutation was not observed in cells treated with the mTOR inhibitor AZD8055. Taken together, these data suggest that TRPML1 activation induces the lipidation and colocalization of ATG8 homologs (e.g., LC3) to lysosomes in a process distinct from autophagosome biogenesis yet dependent on the vATPase. This represents the first example of a small molecule with a defined molecular target that can be used as an inducer of endolysosomal ATG8 conjugation.

### Example 2

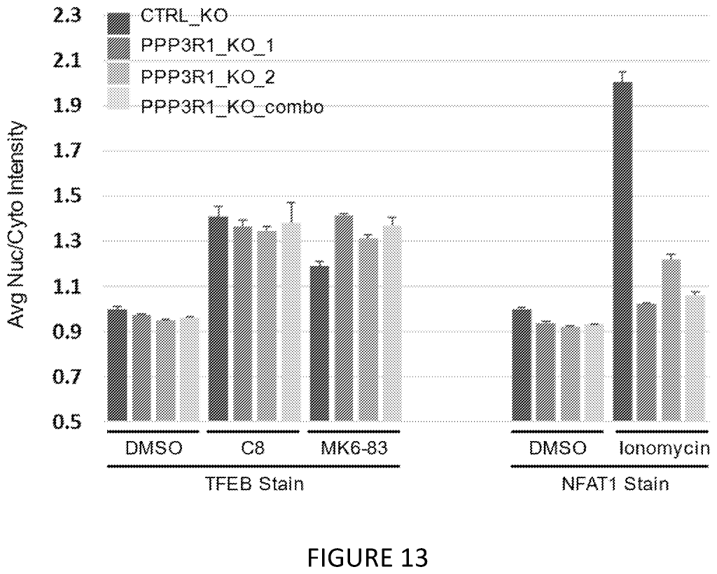

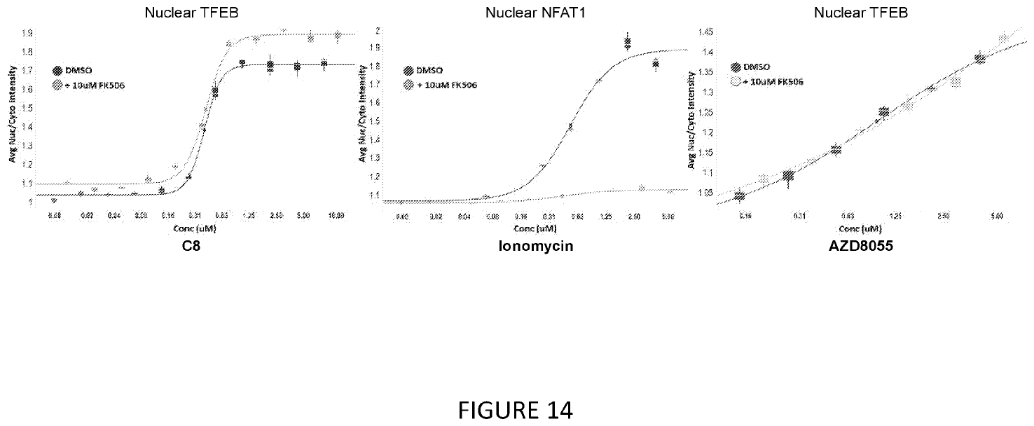

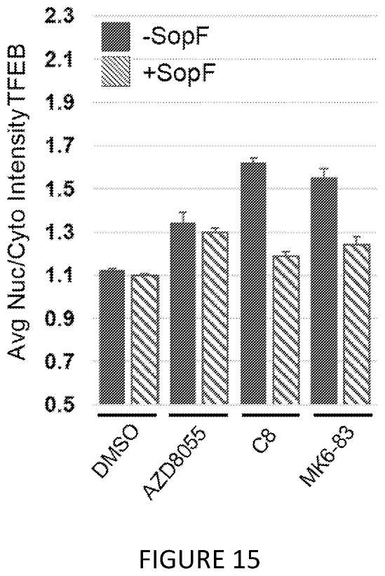

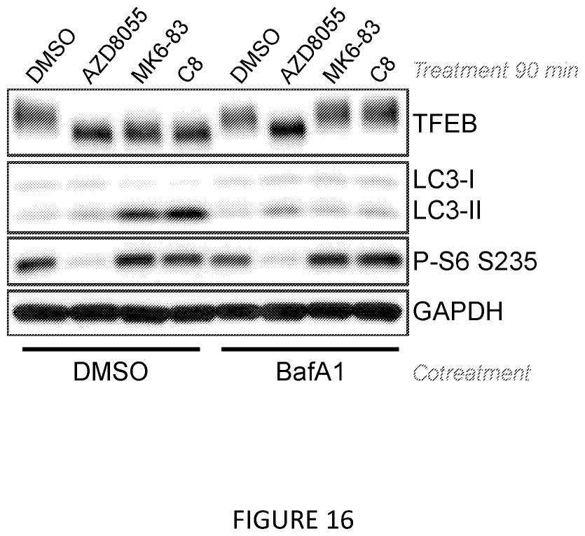

The present Example demonstrates that ATG16L1-dependent ATG8 conjugation to single membranes is important for TFEB activation and lysosomal biogenesis upon changes in lysosomal ion flux. The release of lysosomal calcium through the ion channel TRPML1 is known to result in the nuclear translocation of the transcription factors TFEB and TFE3, presumably due to local activation of the phosphatase Calcineurin (CaN) to dephosphorylate TFEB/TFE3 (14). Using pharmacological inhibitors of CaN and deletion of the essential regulatory subunit PPP3R1 via CRISPR, a role for CaN in the activation of TFEB by TRPML1 agonists was not observed, despite strong inhibition of translocation of the canonical CaN target NFAT1 upon ionomycin treatment (FIG. 13 and FIG. 14). To determine whether TRPML1 agonist-induced ATG8 conjugation is involved in the activation of TFEB, it was determined if expression of SopF, the bacterial effector that blocks ATG16L1 recruitment to the vATPase, could block the nuclear accumulation of TFEB in response to TRPML1 activation. Expression of SopF in cells treated with a TRPML1 agonist inhibited TFEB nuclear accumulation relative to SopF negative controls, whereas SopF expression did not impact TFEB activation upon treatment with AZD8055 (FIG. 15). Additionally, co-treatment with BafA1, which inhibits vATPase activity and lysosomal ATG8 conjugation, prevented TRPML1 agonists (MK6-83 and C8) from activating TFEB, while co-treatment of cells with BafA1 and the mTOR inhibitor AZD8055 did not prevent TFEB activation (FIG. 16). These results are consistent with a role for vATPase in the conjugation of ATG8 proteins to the lysosomal membrane following changes in lumenal ion flux.

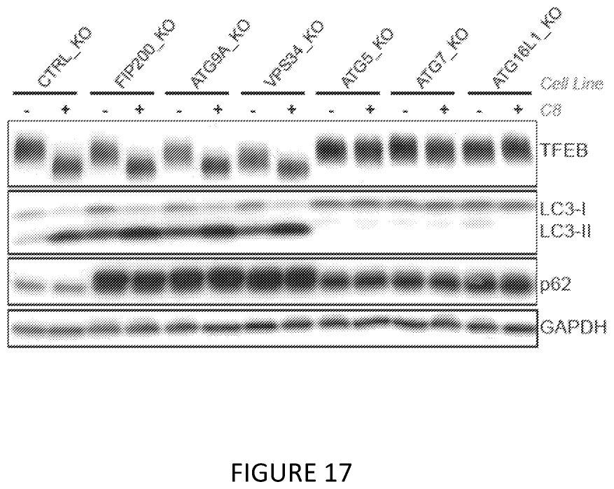

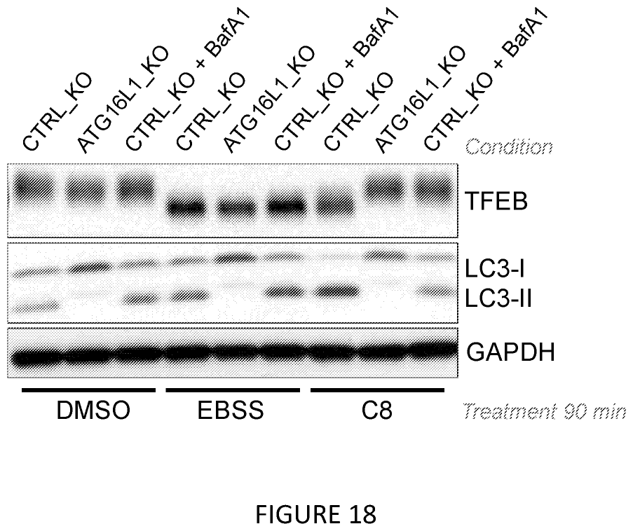

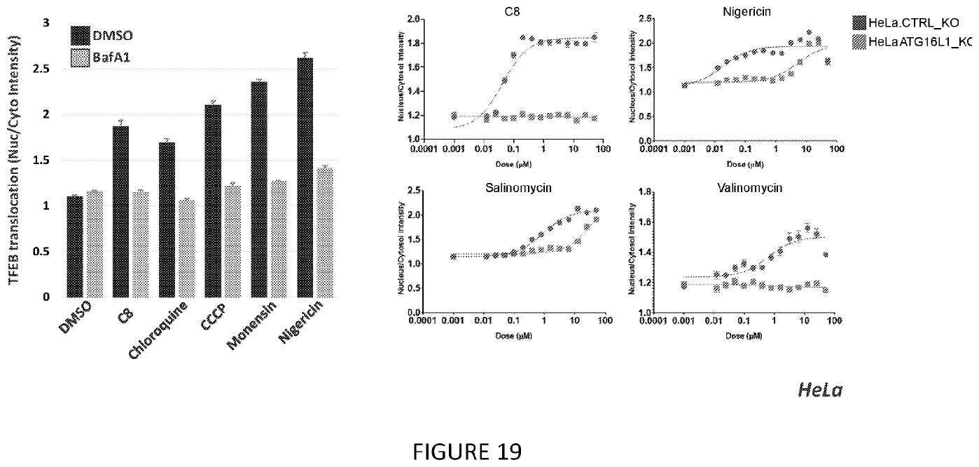

As treatment with BafA1 can also result in depletion of lysosomal Ca²⁺ stores, ATG8 lipidation events were abolished through CRISPR-mediated deletion of ATG16L1, ATG5, or ATG7 (components of the autophagy ATG8 (e.g., LC3) conjugation machinery). Strikingly, it was found that activation of TFEB was completely blocked in cells deficient for ATG8 conjugation (ATG5 KO, ATG7 KO and ATG16L1 KO), but not cells deficient in autophagy through knockout of FIP200, ATG9A, or VPS34 (FIG. 17). Importantly, TFEB activation by a TRPML1 agonist (C8) was sensitive to knockout of ATG16L1 or co-treatment with BafA1, while TFEB activation upon nutrient starvation (EBSS) was insensitive to BafA1 treatment or knockout of ATG16L1 (FIG. 18), suggesting a divergent and specific mechanism of TFEB regulation following alterations in lysosomal ion flux. These results are consistent with a role for vATPase in the conjugation of ATG8 proteins to the lysosomal membrane following changes in lumenal ion flux. Interestingly, other drugs that display ionophore properties and regulate single-membrane ATG8 conjugation (7) were also shown to activate TFEB in an ATG16L-dependent or BafA1-sensitive manner (FIG. 19), suggesting that disruption of lysosomal ion balance may serve as a common trigger to activate TFEB.

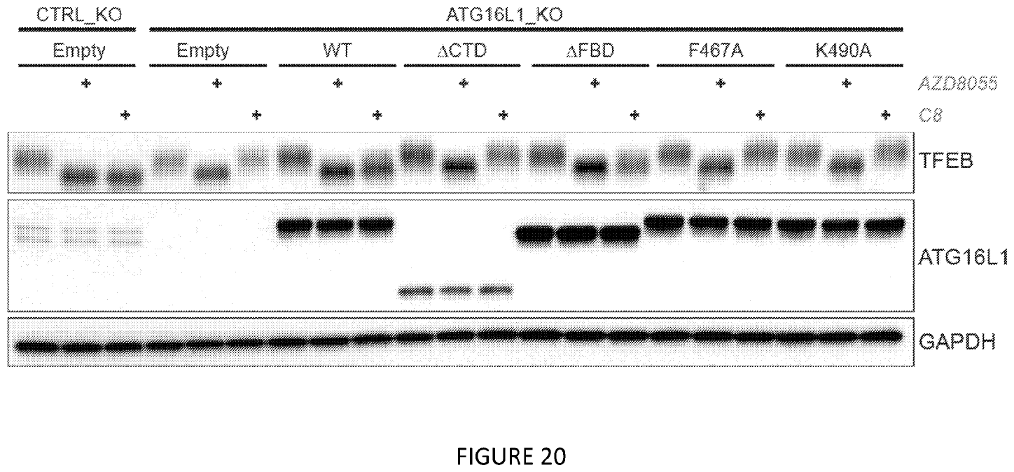

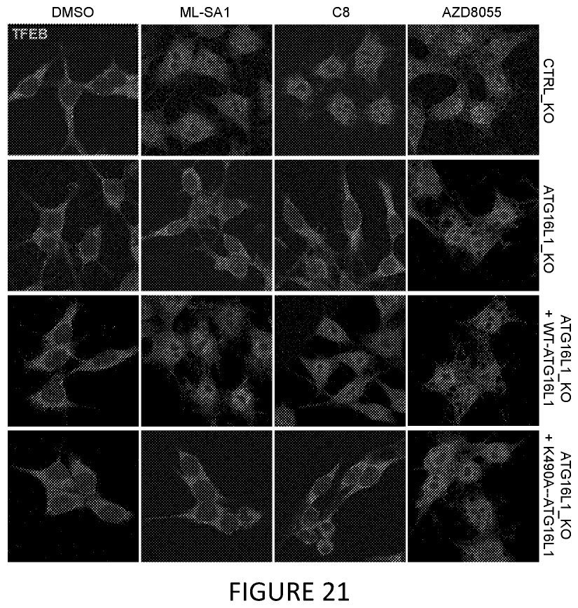

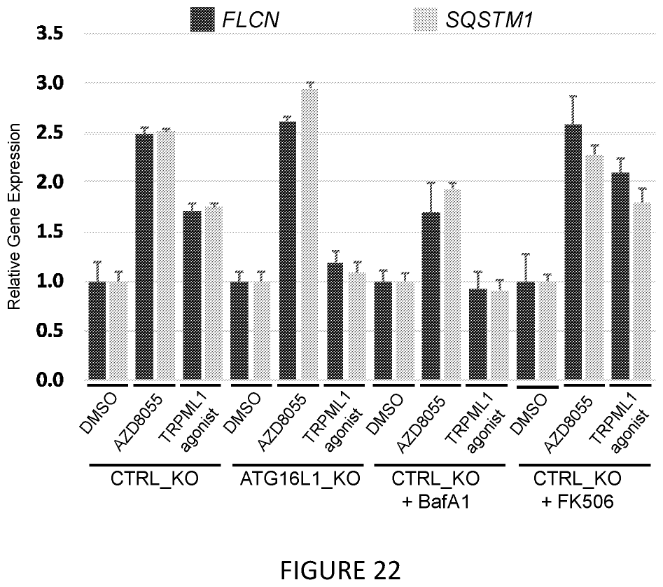

To further isolate the role of single membrane ATG8 conjugation, ATG16L1 knockout cells were reconstituted with several ATG16L1 alleles including a FIP200 binding mutant (ΔFBD) and a C-terminal domain truncation (ΔCTD), which are deficient for autophagosome or single-membrane conjugation, respectively (13). As shown in FIG. 20, activation of TFEB occurred in cells with wild-type ATG16L1 (WT) and ATG16L1-ΔFBD (ΔFBD), but not in ATG16L1-ΔCTD (ΔCTD) cells following treatment with the TRPML1 agonist C8. Additionally, immortalized ATG16L1 KO mouse macrophages reconstituted with WD40 point mutations ATG16L1-F467A (F467A) and ATG16L1-K490A (K490A) did not exhibit TFEB activation in the presence of TRPML1 agonists (e.g., C8 and ML-SA1) (FIG. 21). Importantly, mTOR inhibitor AZD8055 activated TFEB irrespective of ATG16L1 status. These observations demonstrate that the ATG16L1 WD40 domain regulates ATG8 conjugation to the lysosomal membrane and that this is important for TFEB activation by a TRPML1 agonist. To confirm functional TFEB transcriptional activation, we monitored expression of established target genes (e.g., FLCN and SQSTM1) (15) and observed upregulation upon TRPML1 activation in an ATG16L1-dependent, BafA1-sensitive manner. Co-treatment with the calcineurin inhibitor FK506 did not impact gene expression after treatment with AZD8055 or C8 (FIG. 22).

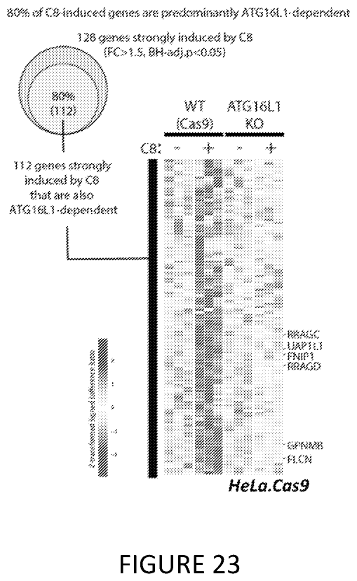

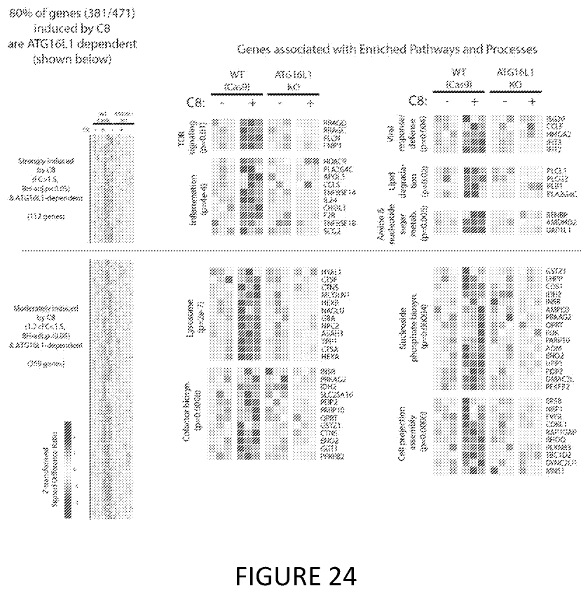

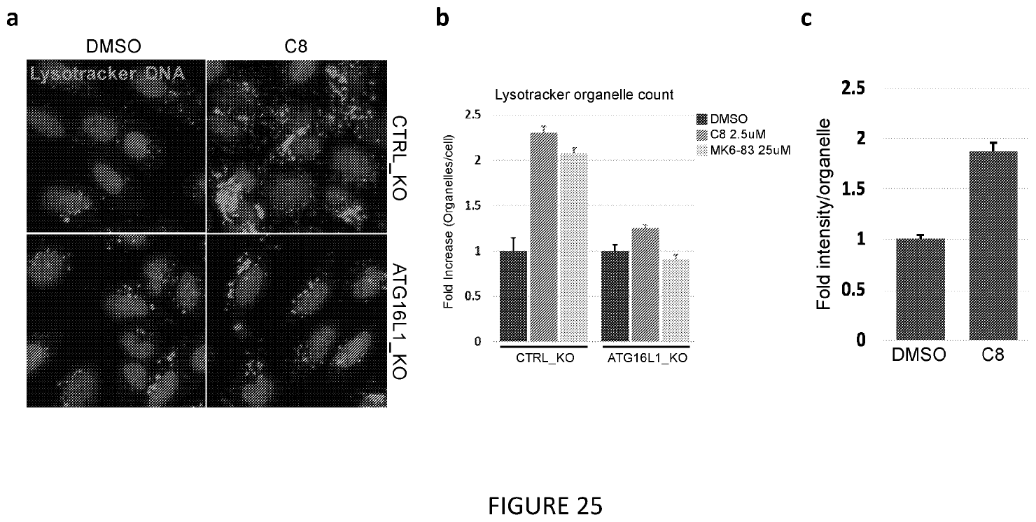

Upon nuclear localization, TFEB serves as the primary transcription factor responsible for lysosomal biogenesis (15). Remarkably, the TRPML1-dependent transcriptomic response was largely dependent on ATG16L1 and included numerous TFEB-target genes involved in lysosomal function (FIG. 23 and FIG. 24). Consistent with this profile, it was found that TRPML1 activation increased both the number and intensity of Lysotracker-positive organelles in an ATG16L1-dependent manner (FIG. 25). Together, these observations demonstrate that following changes in lysosomal ion balance, the WD40 domain in ATG16L1 regulates lysosomal SMAC and that this is required for TFEB activation and lysosomal biogenesis.

### Example 3

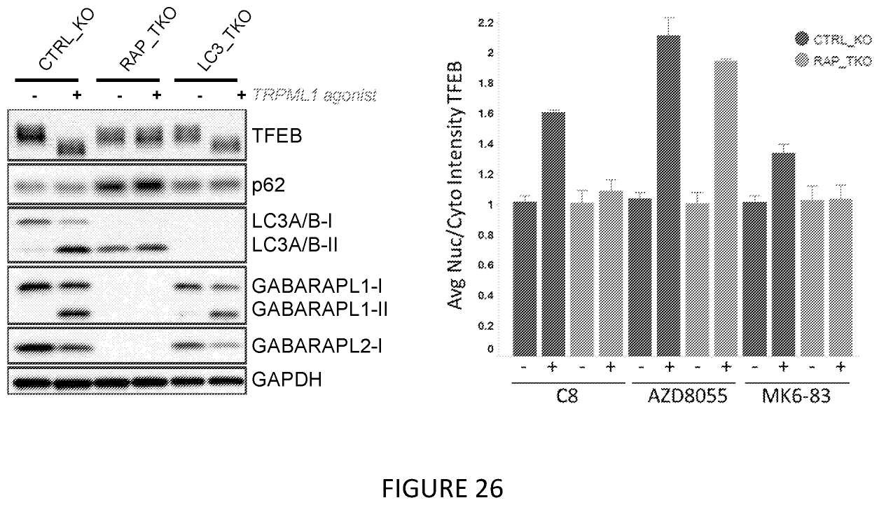

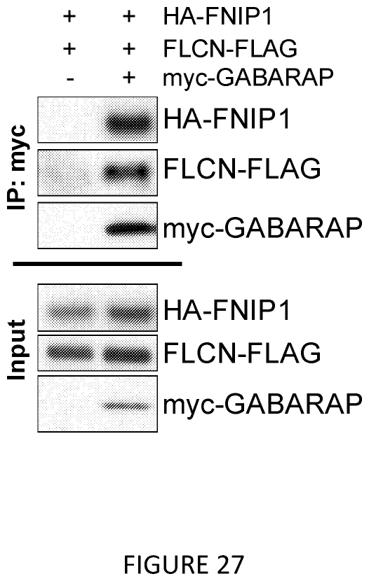

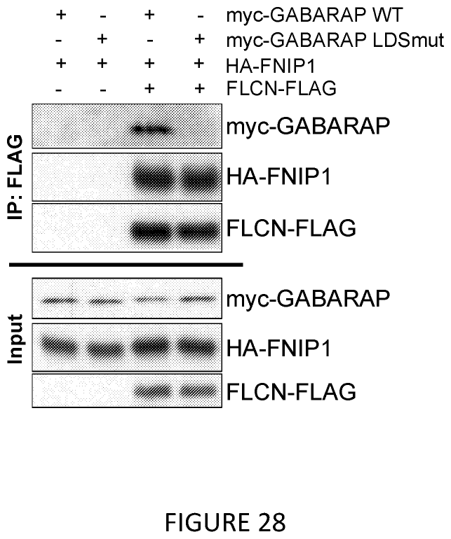

The present Example demonstrates that GABARAP sequesters the FLCN-FNIP1 complex to the lysosomal surface to prevent TFEB cytosolic retention by the RagGTPases. Given the novel role described above for the ATG8 conjugation machinery (e.g., ATG16L1, ATG5, ATG12, ATG7, and ATG3) in the activation of TFEB, the molecular mechanism responsible was then investigated. Mammalian ATG8 homologs include 3 members of the MAP1LC3 family (LC3A/B/C) and 3 members of the GABA type A Receptor-Associated Protein family (GABARAP/L1/L2) (17). Using a combinatorial CRISPR knockout approach, it was found that the GABARAP proteins were specifically involved in the activation of TFEB upon treatment with a TRPML1 agonist (FIG. 26). Analysis of reported binding partners through public interaction databases suggested a unique interaction of GABARAP, but not LC3 proteins, with the TFEB regulators FLCN and FNIP1/2 (18, 19). Co-immunoprecipitation analysis showed that GABARAP could interact with the FLCN-FNIP complex, but that a GABARAP protein comprising a mutation in the LIR domain docking site (LDS) of GABARAP, which is required for LIR-dependent interactions, was unable to pull down the FLCN-FNIP complex (FIG. 27 and FIG. 28).

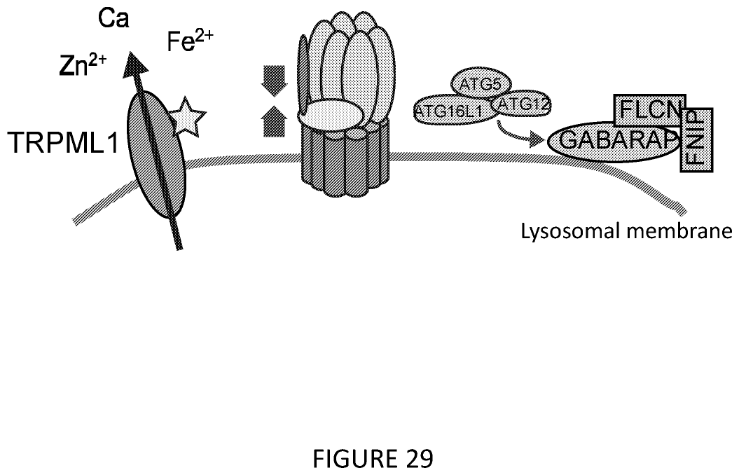

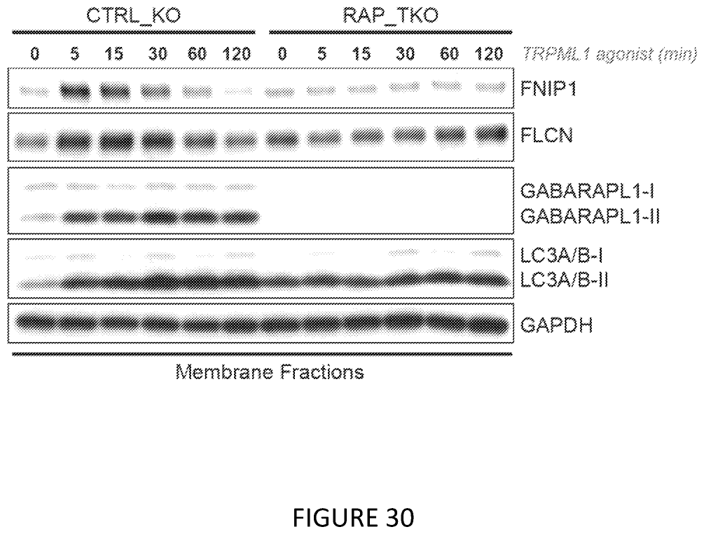

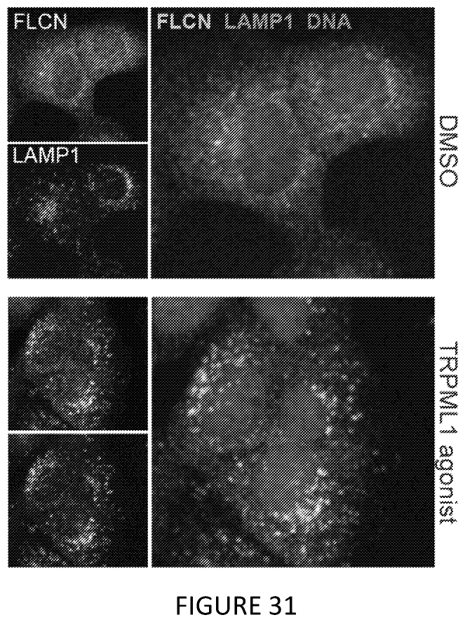

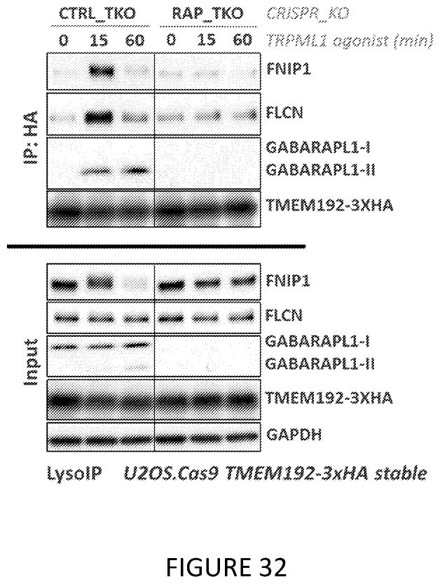

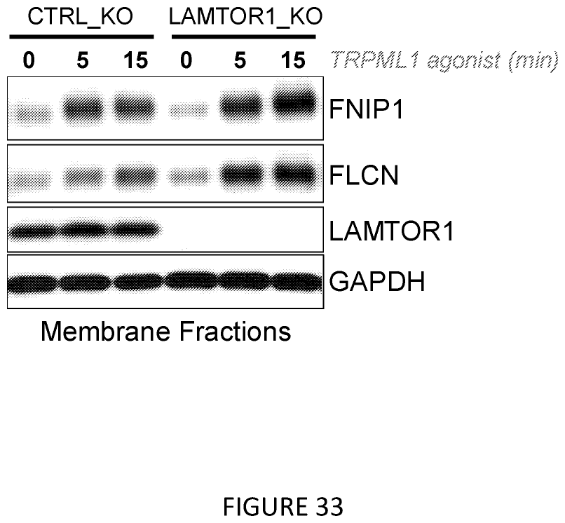

It was hypothesized that in response to changes in lysosomal ion flux, the direct conjugation of GABARAPs to lysosomal membranes could re-distribute the FLCN/FNIP complex from the cytosol to the lysosomal membrane (FIG. 29). Indeed, following TRPML1 activation a rapid and robust increase in membrane-associated FLCN and FNIP1 was observed, and this was dependent on expression of GABARAP proteins (FIG. 30). Additionally, as shown in FIG. 31, cells treated with a TRPML1 agonist exhibited enhanced co-localization of FLCN with the lysosomal protein LAMP1 as compared to control. To confirm recruitment specifically to lysosomal membranes, cells were engineered to express the 3XHA-TMEM192 protein, a lysosome specific marker that can be used as a handle to rapidly purify lysosomes (36). Lysosome purification revealed a robust recruitment of FLCN and FNIP1 within 15 minutes of TRPML1 agonist treatment, which involved GABARAP proteins (FIG. 32). Lysosomal localization of FLCN also occurs upon nutrient starvation, where FLCN specifically binds to RagA^{GDP} and forms the inhibitory lysosomal folliculin complex (LFC) (21, 22). However, in cells deficient for the Ragulator complex component LAMTOR1, which is part of the LFC, TRPML1 activation still induced FLCN membrane recruitment, indicating that the GABARAP-dependent sequestration of FLCN/FNIP1 was a process distinct from LFC formation (FIG. 33).

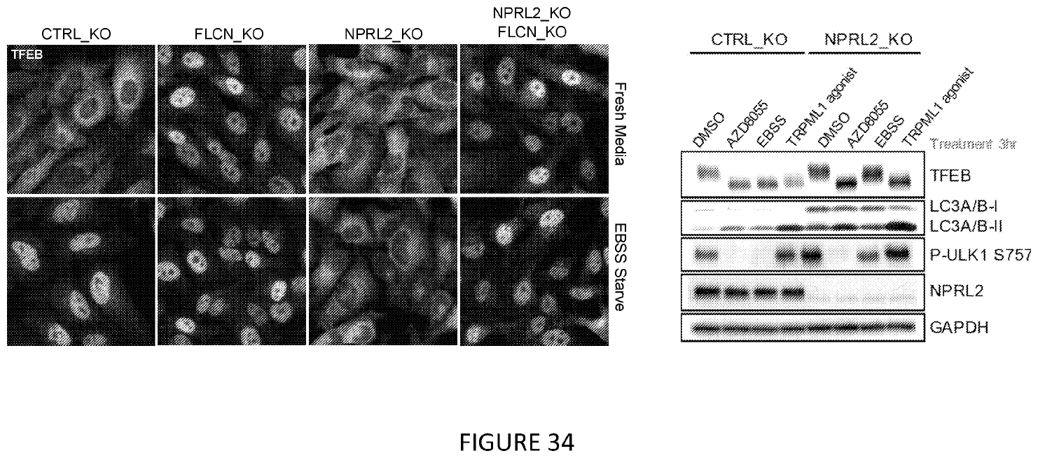

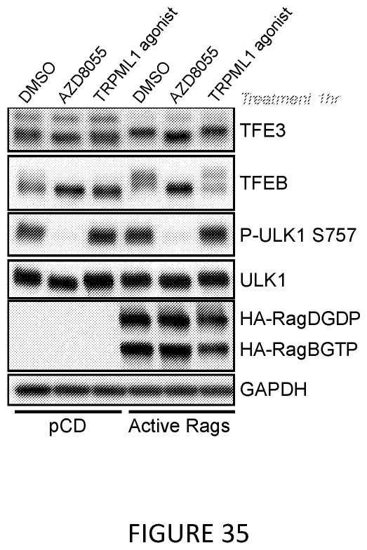

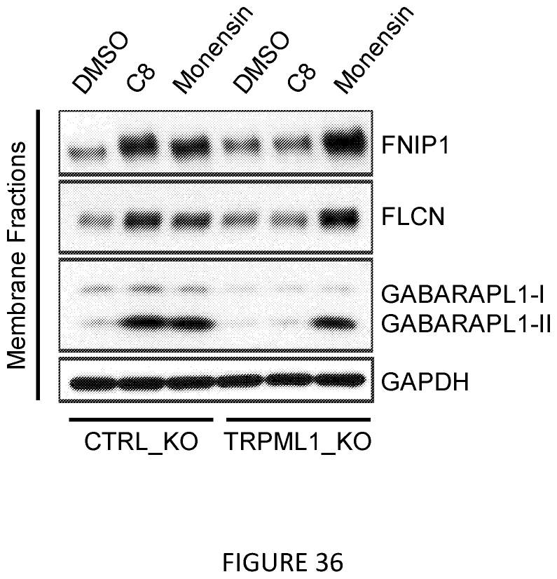

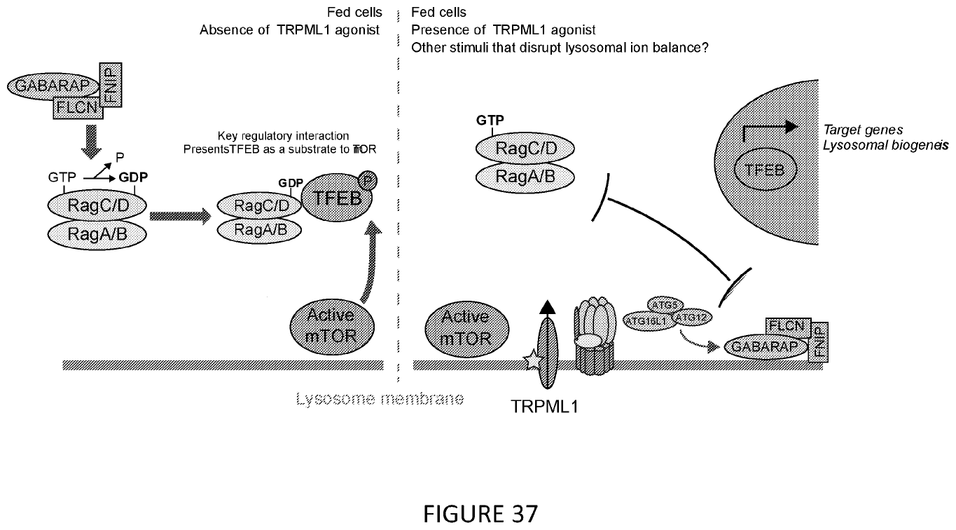

Analogous to the inhibitory role of LFC formation on FLCN-FNIP GAP activity (21, 22), it was hypothesized that GABARAP-dependent recruitment of FLCN to the lysosome would also inhibit GAP activity toward RagC/RagD. This would suggest that FLCN-FNIP1 exerts its GAP function toward RagC/D away from the lysosomal surface, thus promoting the RagC/D^{GDP}-bound state and subsequent RagC/D-binding dependent TFEB cytosolic retention (23, 24). Indeed, RagGTPase dimers have been shown to interact dynamically with the lysosome under fed conditions (25). To test this, NPRL2 KO cells were used, which have constitutive RagA/B^{GTP} and defective lysosomal localization of FLCN upon starvation (26). Knockout of FLCN resulted in complete nuclear translocation of TFEB under nutrient rich conditions in both wild-type and NPRL2 KO cells, supporting the model that FLCN-FNIP1 acts as a GAP for RagC/D away from the lysosomal surface to regulate TFEB (FIG. 34). Additionally, whereas TFEB cannot be activated upon starvation in NPRL2 KO cells due to failed formation of the LFC, cells treated with a TRPML1 agonist retained the ability to activate TFEB, consistent with inhibition of continued GAP activity of FLCN toward RagGTPases (FIG. 34). However, expression of RagGTPases locked in the active state (RagB^{Q99L}/RagD^{S77L}), which are no longer regulated by FLCN-FNIP1, suppressed the mobility shift of TFEB and its homolog TFE3 following TRPML1 activation but not with AZD8055 (FIG. 35). The GABARAP-dependent sequestration of FLCN can also occur independently of TRPML1 (FIG. 36), suggesting that this mechanism of TFEB regulation could be coupled broadly to other stimuli that result in conjugation of GABARAP proteins to membrane compartments other than the lysosome. Collectively, the activation of TFEB upon alteration of lysosomal ion contents appears to rely on the GABARAP-dependent tethering of the FLCN-FNIP1 complex to the lysosomal surface where it is unable to exert GAP activity towards RagC/D. This results in a stabilization of the RagC/D^{GTP} state and liberation of TFEB from this complex, allowing for its translocation into the nucleus and TFEB-dependent lysosomal biogenesis (FIG. 37).

### Example 4

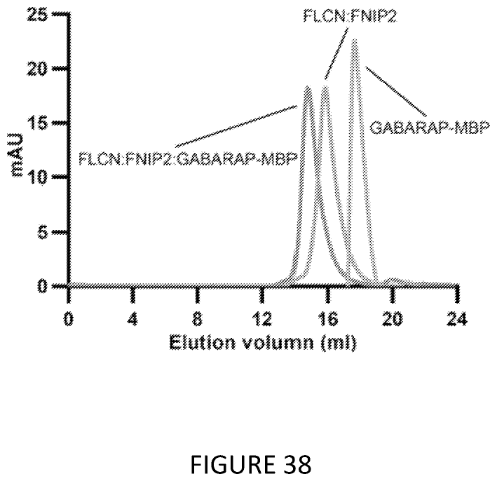

This example elucidates a novel binding site for GABARAP on FNIP proteins and identifies key amino acid residues involved in the selectivity of ATG8 family proteins for LIR domains. To map the binding site of GABARAP within the FLCN-FNIP complex, each protein was made recombinantly and purified using standard techniques. GABARAP bound the FLCN-FNIP2 complex in vitro and could be co-purified over a sizing column (FIG. 38).

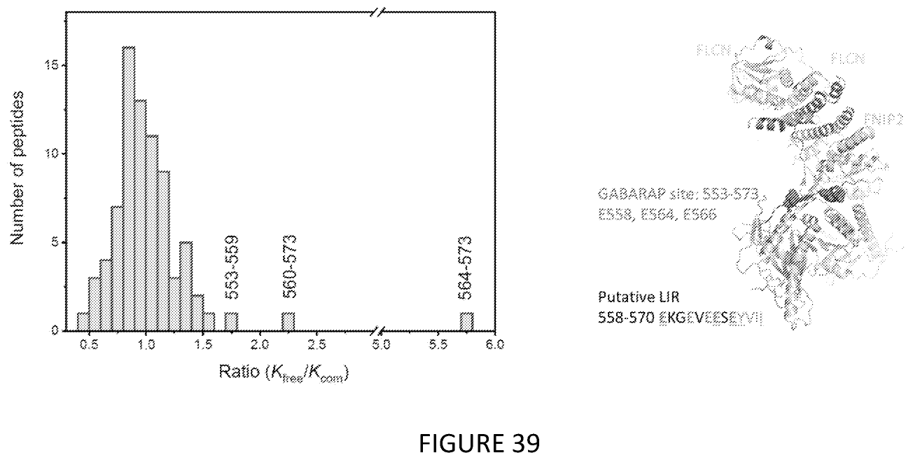

A chemical footprinting approach was undertaken using the GEE labeling technique (37). In this method, a covalent probe is incubated with a protein of interest. The probe will link to aspartic acid and glutamic acid residues and this pattern can be analyzed upon protein digestion and loading on a mass spectrometer. For the present experiments, a labeling pattern was established for the FLCN-FNIP2 complex. The FLCN-FNIP2 complex was then incubated with GABARAP and GEE labeling was performed again, to determine which residues were protected from binding, and therefore constitute a binding site between GABARAP and FLCN-FNIP2. As shown in FIG. 39, a specific region in FNIP2 appeared to be protected as evidence by the increased labeling in the free sample (FLCN/FNIP alone) versus the complex sample (FLCN/FNIP+GABARAP). The ratio of Kfree/Kcomplex for each residue was calculated and a ratio greater than 1.6 was deemed significant for protection. The specific protected region is highlighted in red within the structural representation of the FLCN/FNIP complex. This specific sequence contained a previously unreported LIR domain (YVVI) that is conserved in both FNIP1 and FNIP2.

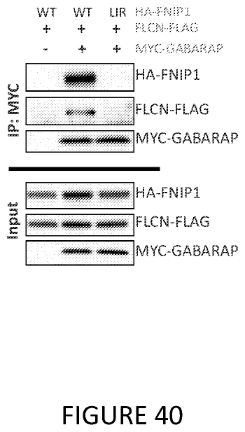

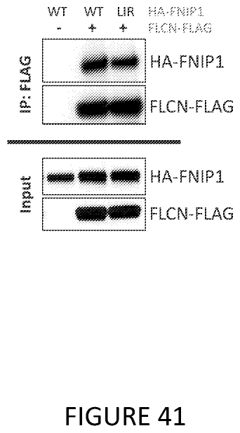

To confirm that the identified region of FNIP mediates the interaction of GABARAP with the FLCN-FNIP complex, point mutations in the LIR domain of FNIP1 were generated (YVLV>AVLA). Co-immunoprecipitation experiments confirmed that GABARAP required the LIR domain YVLV to interact with the FLCN-FNIP1 complex (FIG. 40). Mutations in the FNIP1 LIR did not affect the interaction between FNIP1 and FLCN (FIG. 41).

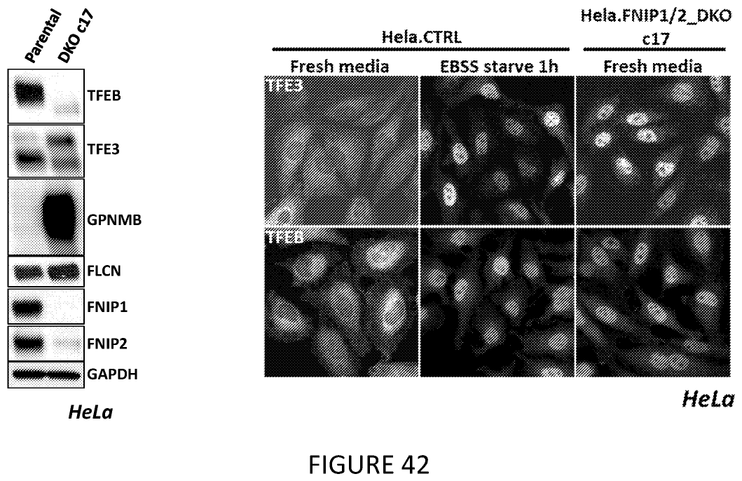

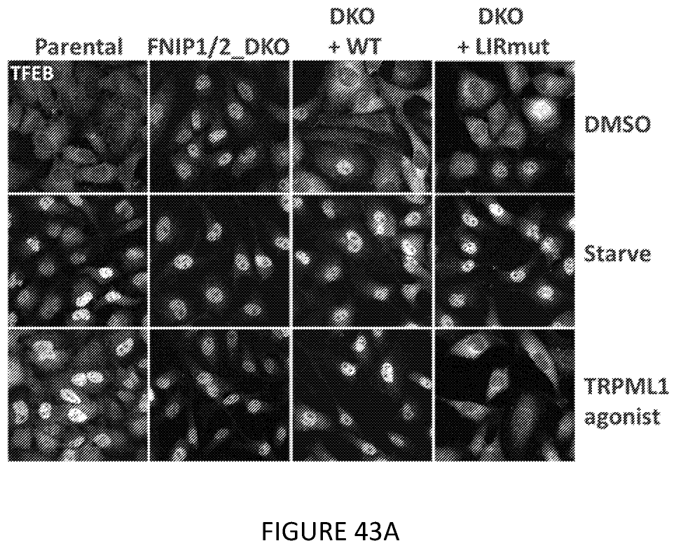

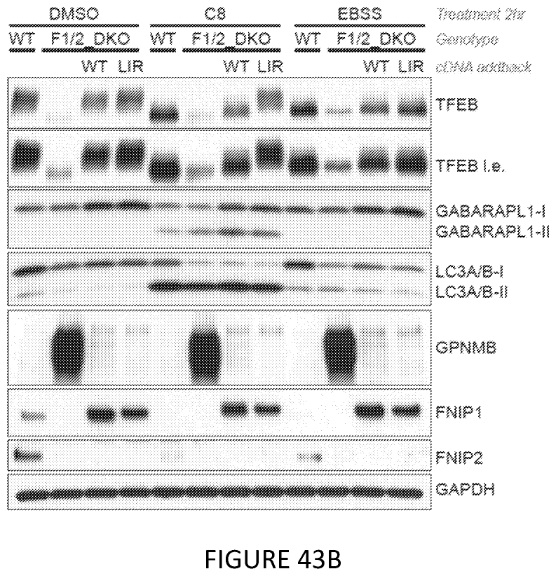

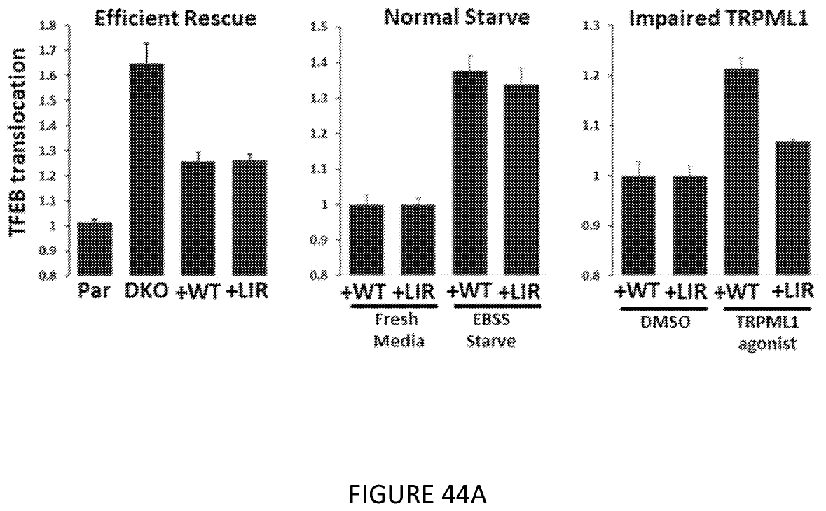

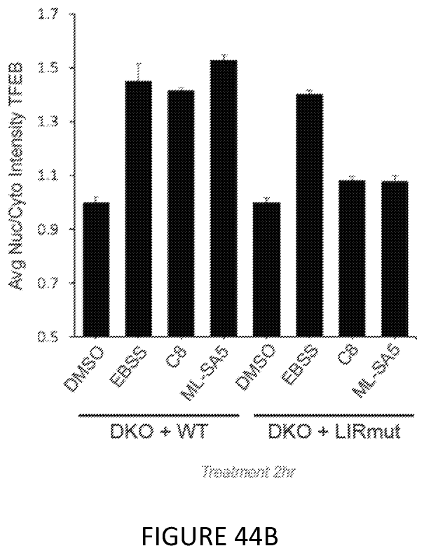

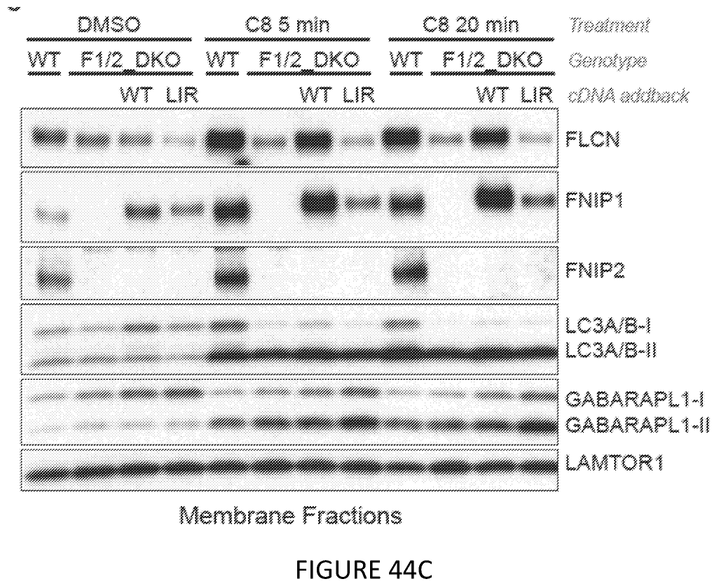

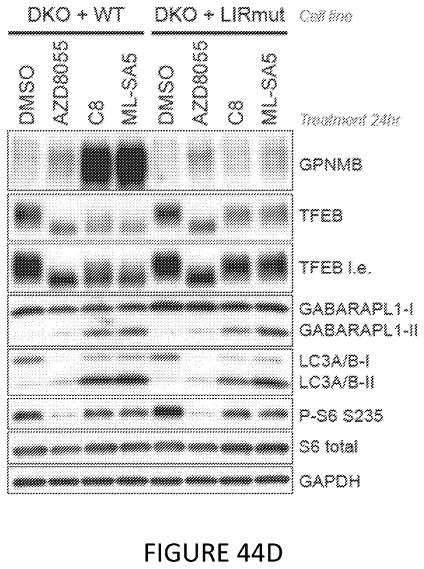

FNIP1/FNIP2 double knockout cells in a HeLa background were then created. FNIP1/2 DKO resulted in a complete loss of FLCN GAP activity and constitutive activation of TFEB and TFE3 transcription factors, as evidenced by persistent nuclear localization and increased expression of the TFEB transcriptional target GPNMB (FIG. 42). These cells were then reconstituted with either WT-FNIP1 or LIRmut-FNIP1. Pooled populations of DKO cells expressing FNIP1 showed partial rescue of constitutive TFEB/TFE3 nuclear localization. When these cells were starved of nutrients, TFEB and TFE3 were activated normally irrespective of the FNIP1 variant expressed. However, when treated with a TRPML1 agonist, TFEB nuclear localization was only observed in the cells expressing WT-FNIP1 and not in cells harboring a LIR mutant FNIP1 variant (FIG. 43A and FIGS. 44A-B). As shown in FIG. 43B, reconstitution of FNIP1/2 double knockout (DKO) cells with either WT or LIR (LIR-mutant Y583A/V586A) FNIP1 revealed a functional requirement of GABARAP interaction for TRPML1 agonist, but not EBSS, activation of TFEB. Upon chronic treatment with a TRPML1 agonist, the functional TFEB transcriptional response was measured by protein levels of the target gene GPNMB. GPNMB expression was completely blocked in FNIP1-LIR mutant cells (FIG. 44D). GPNMB protein levels were also largely suppressed upon AZD8055 treatment despite robust TFEB activation. This highlights how concurrent inhibition of protein translation may minimize the effective scope of the TFEB transcriptional activation (FIG. 44D). ATG8 conjugation was not altered by modulation of FNIP1 (FIG. 43B). These findings confirms that the GABARAP-dependent re-localization of the FLCN-FNIP complex through direct interaction with FNIP proteins is important for the activation of TFEB upon changes in lysosomal ion flux.

### Example 5

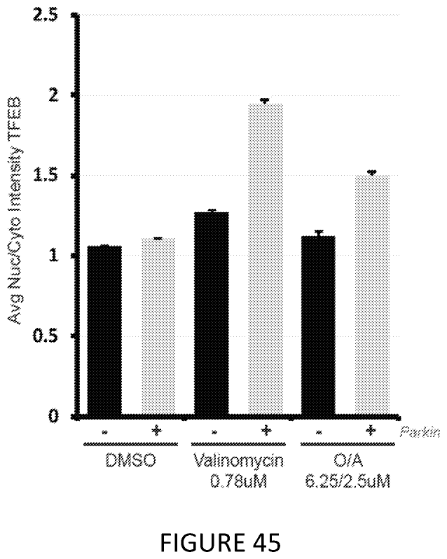

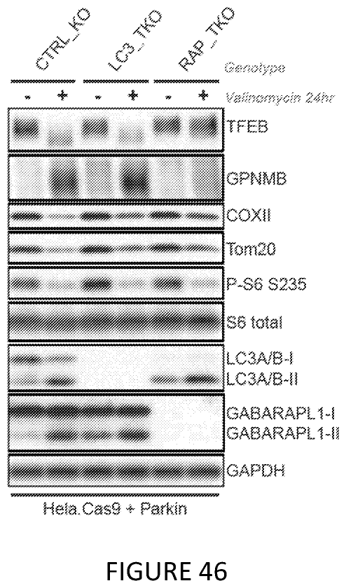

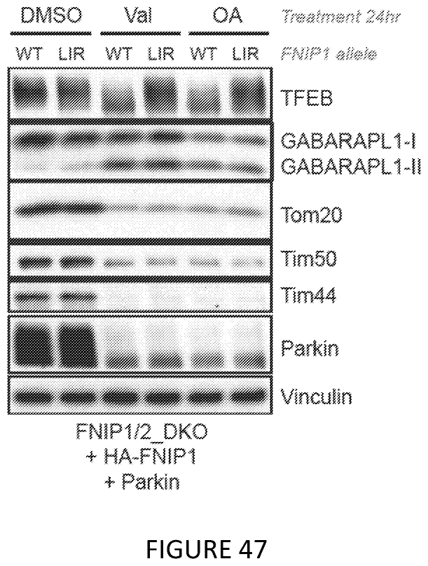

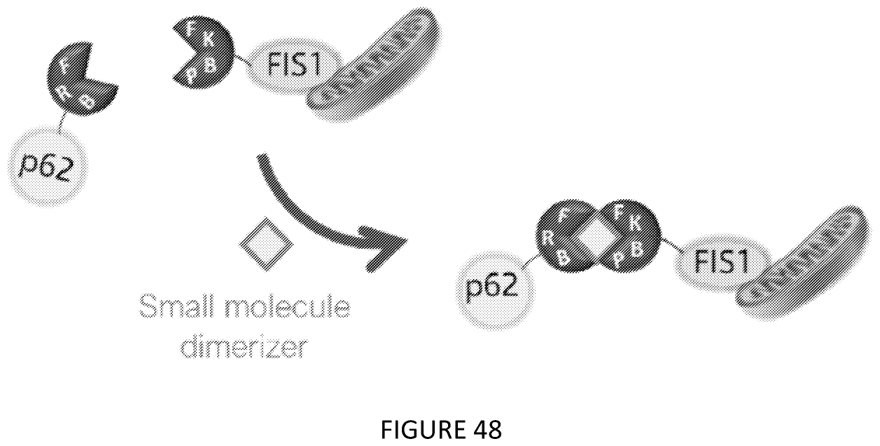

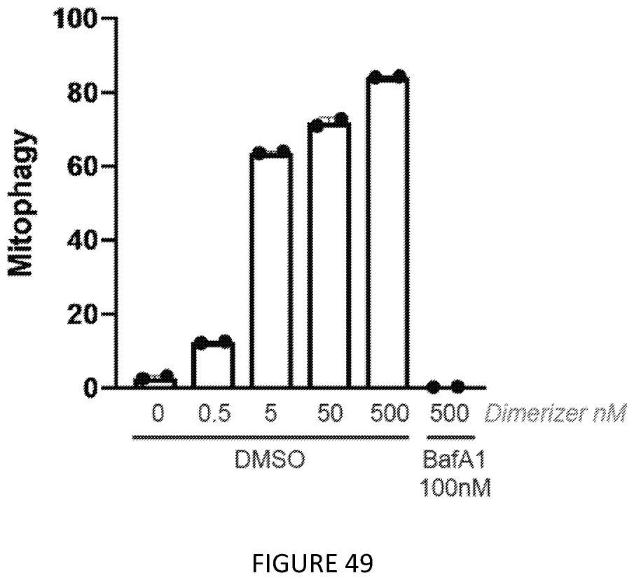

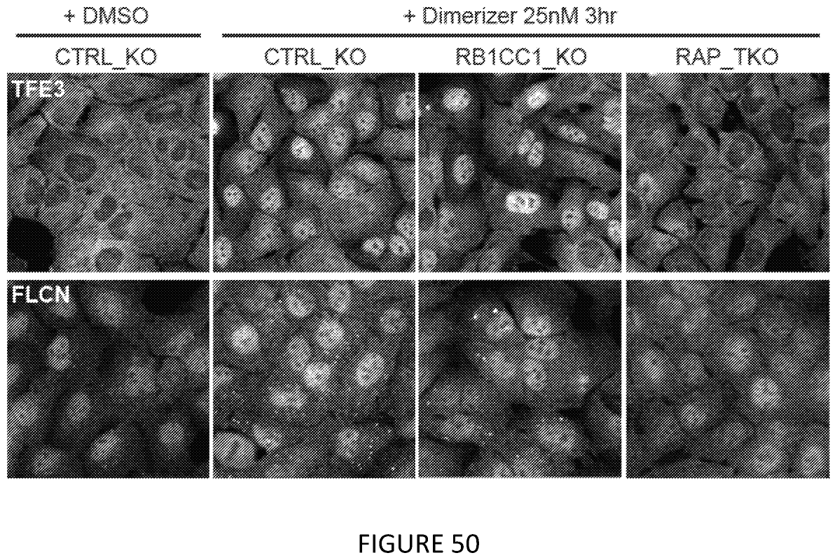

This example examines the concept that any instance where GABARAP proteins are conjugated to subcellular membranes might result in TFEB activation via the high affinity sequestration of FLCN/FNIP. Thus, distinct forms of selective autophagy, mitophagy and xenophagy were examined. It has been shown that TFEB activation occurs during parkin-dependent mitophagy (47), and, as shown in FIGS. 45 and 46, this activation involved GABARAP proteins. Furthermore, TFEB activation was defective in cells stably expressing LIR-mutant FNIP1, confirming that GABARAP-dependent re-localization of FLCN to mitochondria mechanistically links TFEB activation to mitophagy (FIG. 47). Interestingly, an earlier study observed that FLCN and FNIP could localize to mitochondria upon depolarization (48) and the TFEB activation mechanism revealed herein indicates the relevance of this. Importantly, using a proximity-regulated mitophagy system, where mitophagy was measured using the Keima fluorescence shift assay (49) (FIGS. 48 and 49) the GABARAP-dependence of TFE3 translocation and FLCN redistribution independent of mitochondrial uncouplers was confirmed (FIG. 50).

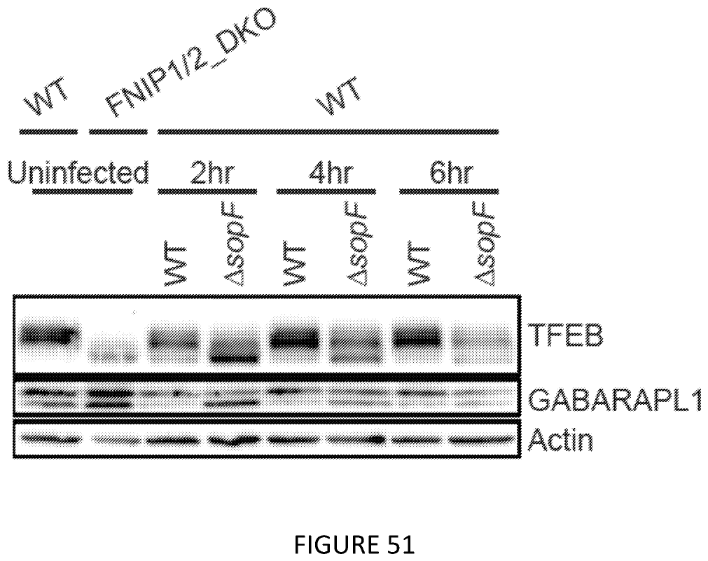

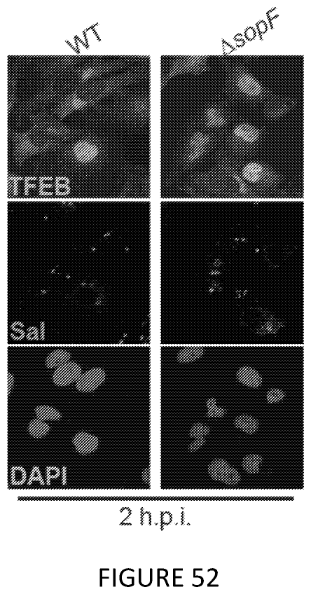

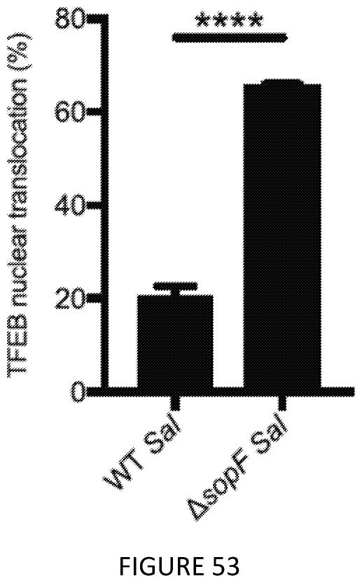

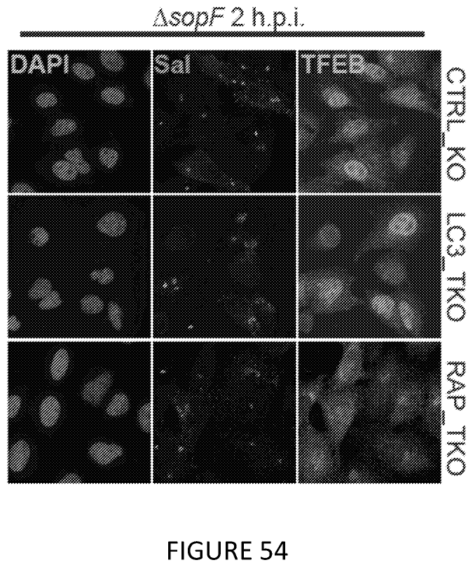

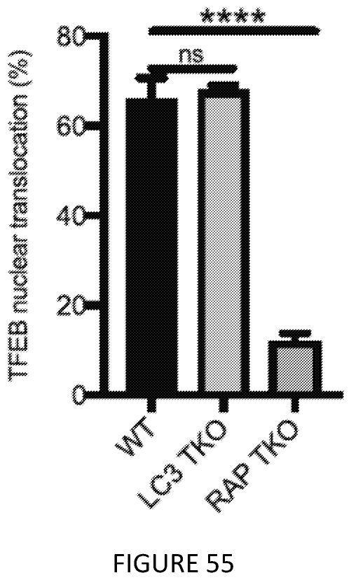

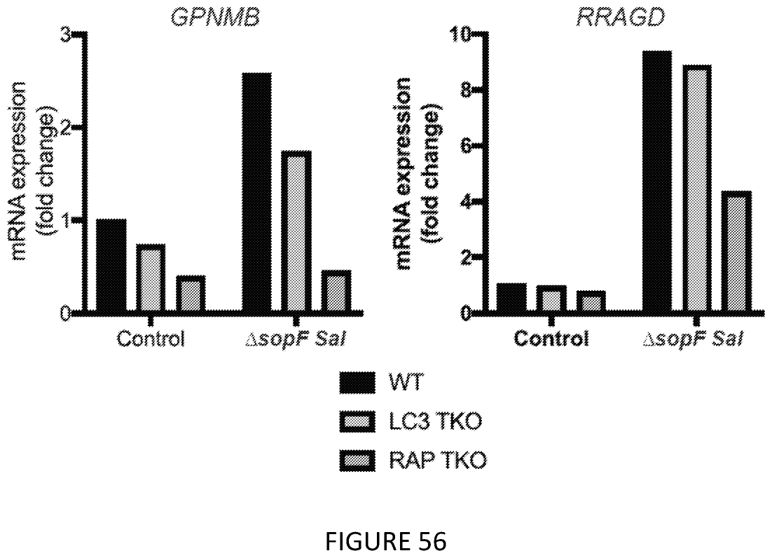

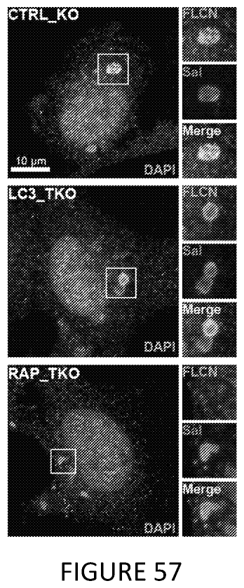

Finally, a Salmonella infection model of xenophagy was used to determine if TFEB activation was regulated by GABARAP-mediated FLCN sequestration. A portion of Salmonella enterica serovar Typhimurium (S. Typhimurium) are rapidly targeted by the autophagy machinery and become decorated with ATG8 homologs (50). It was recently discovered that S. Typhimurium antagonize the ATG8 response through the bacterial effector SopF (8). It was hypothesized that if ATG8 proteins were involved in TFEB activation, ΔsopFS. Typhimurium would show a greater TFEB activation than WT due to increased ATG8 conjugation. Indeed, ΔsopFS. Typhimurium produced a robust activation of TFEB that occurred in a higher percentage of cells for a longer duration of time post-infection (FIGS. 51-53). Importantly, TFEB activation was blunted by deletion of GABARAP family members (RAP_TKO), but was not impacted by knockout of LC3 isoforms (LC3_TKO) (FIGS. 54-56). The localization of FLCN was also examined and a striking re-localization of FLCN was found to coat the S. Typhimurium Salmonella-containing vacuole membrane (FIG. 57). This re-localization involved GABARAP proteins, indicating that GABARAP-dependent sequestration of FLCN to the Salmonella vacuoles results in TFEB activation upon infection (FIG. 57). Taken together, these mitophagy and xenophagy examples of selective autophagy highlight that GABARAP-dependent sequestration of FLCN to distinct cellular membranes may serve as a universal mechanism to couple activation of TFE3/TFEB transcription factors to the initiation of selective autophagy.

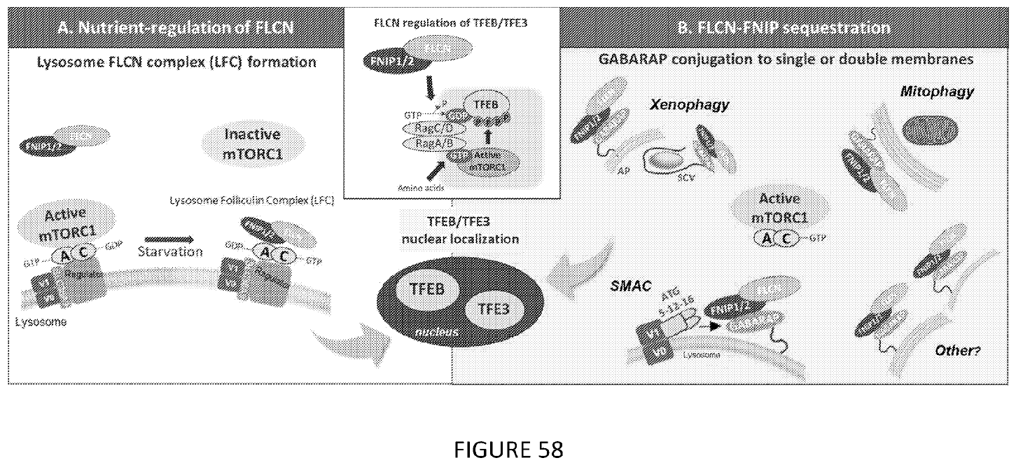

As shown in FIG. 58, the FLCN-FNIP GAP complex critically regulates the mTOR-dependent phosphorylation and cytosolic retention of the TFEB/TFE3 transcription factors by promoting the GDP-bound state of RagC/D. GDP-bound RagC/D directly binds to and presents TFEB/TFE3 as a substrate to mTOR (center inset), as described previously. During nutrient starvation (section A), recruitment of FLCN-FNIP to the lysosomal membrane helps form the lysosomal folliculin complex (LFC), which has reduced GAP activity towards RagC/D. This is coincident with mTORC1 inhibition. Independently of LFC formation, GABARAP proteins bind directly to the FLCN-FNIP complex and sequester it at diverse intracellular membranes (section B). This membrane recruitment is needed for TFEB activation in response to endolysosomal ion disruption and forms of selective autophagy (xenophagy and mitophagy). This suggests that FLCN-FNIP regulates cytosolic RagC-GTP and its sequestration on intracellular membranes reduces access to this substrate, allowing for nuclear retention of TFEB/TFE3 due to impaired Rag binding. Unlike nutrient regulation of FLCN, this novel TFEB activation pathway is permissive with mTORC1 activity. Subcellular redistribution of the FLCN-FNIP complex to both single and double membranes serves to broadly coordinate lysosomal capacity with homeostasis and perturbations within the endolysosomal network.

In summary, described herein is a previously unrecognized molecular mechanism orchestrated upon lysosomal single-membrane ATG8 conjugation (SMAC). Changes in lysosomal ion levels revealed a novel regulatory input to TFEB nuclear localization that is independent of nutrient status but involves the ATG5-ATG12-ATG16L1 conjugation machinery. SMAC allows for sensitive detection of dysfunction within the endolysosomal pathway, possibly as part of a host-pathogen response. The conjugation of ATG8 homologs to endolysosomal membranes is often inhibited by pathogen virulence factors, for example SopF of S. typhimurium (8), CpsA of M. tuberculosis (27), and RavZ of Legionella (28). Recently, it has been proposed that disruption of the phagosomal ion gradient triggered ATG8 modification of the ΔSopF S. typhimurium-containing vacuole and that this precedes vacuole rupture and xenophagy (8). The lysosomal SMAC-TFEB activation mechanism described herein is the first to assign a function for single membrane ATG8 conjugation. Indeed, induction of SMAC may serve to couple TFEB-dependent transcription of cytoprotective/antimicrobial genes (29) and lysosomal biogenesis to limit pathogen infection. Furthermore, inducers of SMAC can evoke markers of autophagosome biogenesis (30) and the latter has recently been shown with TRPML1 activators (31). Finally, these data reveal a novel regulatory mechanism of the FLCN-FNIP tumor suppressor complex, as a dominant regulator of TFEB/TFE3 activity (19, 32). The GABARAP-dependent sequestration of FLCN can also occur independently of TRPML1 (FIG. 36), suggesting that this mechanism of TFEB regulation could be coupled broadly to other stimuli that result in conjugation of GABARAP proteins to membrane compartments other than the lysosome. GABARAP function as a signaling modulator represents a novel role for ATG8 proteins outside of substrate degradation and vesicle fusion. Further understanding of how this ubiquitin-like homeostatic response is modulated under pathological conditions may offer new opportunities for therapeutics that impact the endolysosomal system.

### REFERENCES

1. Kaur, J., and J. Debnath, Autophagy at the crossroads of catabolism and anabolism. Nat Rev Mol Cell Biol. 16, 461-72 (2015).
2. Florey, O., et al., Autophagy machinery mediates macroendocytic processing and entotic cell death by targeting single membranes. Nat Cell Biol. 13, 1335-43 (2011).
3. Martinez, J. S., et al., Microtubule-associated protein 1 light chain 3 alpha (LC3)-associated phagocytosis is required for the efficient clearance of dead cells. Proc. Nat. Acad. Sci. 108, 17396-401 (2011).
4. Jacquin, E., et al., Pharmocological modulators of autophagy activate a parallel noncanonical pathway driving unconventional LC3 lipidation. Autophagy. 13, 854-867 (2017).
5. Heckmann, B. L., et al., LC3-associated endocytosis facilitates β-amyloid clearance and mitigates neurodegeneration in murine Alzheimer's disease. Cell. 178, 536-51 (2019).
6. Galluzzi, L., and D. R, Green, Autophagy-independent functions of the autophagy machinery. Cell. 177, 1682-99 (2019).
7. Florey, O., et al., V-ATPase and osmotic imbalances activate endolysosomal LC3 lipidation. Autophagy. 11, 88-99 (2015).
8. Xu, Y., et al., A bacterial effector reveals the V-ATPase-ATG16L1 axis that initiates xenophagy. Cell. 178, 1-15 (2019).
9. Chen, C-C., et al., A small molecule restores function to TRPML1 mutant isoforms responsible for mucolipidosis type IV. Nat Comm., 5, 1-10 (2014).
10. Shen, D., et al., Lipid storage disorders block lysosomal trafficking by inhibiting a TRP channel and lyosomal calcium release. Nat Comm. 3, 1-11 (2012).
11. Liang, C., Piperazine derivatives as trpml modulators. WO 2018/005713 A1. Jan. 4 (2018).
12. Chresta, C. M., et al., AZD8055 is a potent, selective, and orally bioavailable ATP-competitive mammalian target of rapamycin kinase inhibitor with in vitro and in vivo antitumor activity. Cancer Res. 70, 288-98 (2010).
13. Fletcher, K., et al., The WD40 domain of ATG16L1 is required for its non-canonical role in lipidation of LC3 at single membranes. EMBO J. 37, e97840 (2018).
14. Medina, D. L., et al., Lysosomal calcium signaling regulates autophagy through calcineurin and TFEB. Nat Cell Biol. 17, 288-99 (2015).
15. Sardiello, M., et al., A gene network regulating lysosomal biogenesis and function. Science. 325, 473-7 (2009).
16. Rai, S., et al., The ATG5-binding and coiled coil domains of ATG16L1 maintain autophagy and tissue homeostasis in mice independently of the WD domain required for LC3-associated phagocytosis. Autophagy. 15, 599-612 (2019).
17. Johansen, T., and T., Lamark. Selective autophagy: ATG8 family proteins, LIR motifs and cargo receptors. J Mol Biol. 432, 80-103 (2020).
18. Dunlop, E. A., et al. FLCN, a novel autophagy component, interacts with GABARAP and is regulated by ULK1 phosphorylation. Autophagy. 10, 1749-1760 (2014).
19. Hong, S. B., et al., Inactivation of the FLCN tumor suppressor gene induces TFE3 transcriptional activity by increasing its nuclear translocation. PLoS One. 5, e15793 (2010).
20. Petit, C. S., et al., Recruitment of folliculin to lysosomes supports the amino acid-dependent activation of Rag GTPases. J Cell Biol. 202, 1107-1122 (2013).
21. Lawrence, R. E., et al., Structural mechanism of a Rag GTPase activation checkpoint by the lysosomal folliculin complex. Science. 366, 971-977 (2019).
22. Shen, K., et al., Cryo-EM structure of the human FLCN-FNIP2-Rag-Ragulator complex. Cell. 179, 1319-1329 (2019).
23. Settembre, C., et al., A lysosome-to-nucleus signalling mechanism senses and regulates the lysosome via mTOR and TFEB. EMBO J. 31, 1095-1108 (2012).
24. Martina, J. A., and R., Puertollano. Rag GTPases mediate amino acid-dependent recruitment of TFEB and MITF to lysosomes. J Cell Biol. 200, 475-91 (2013).
25. Lawrence, R. E., et al., A nutrient-induced affinity switch controls mTORC1 activation by its Rag GTPase-Ragulator lysosomal scaffold. Nat Cell Biol. 20, 1052-1063 (2018).
26. Meng, J., and S. M., Ferguson. GATORI-dependent recruitment of FLCN-FNIP to lysosomes coordinates Rag GTPase heterodimer nucleotide status in response to amino acids. J Cell Biol. 217, 2765-2776 (2018).
27. Koster, S., et al., Mycobacterium tuberculosis is protected from NADPH oxidase and LC3-associated phagocytosis by the LCP protein CpsA. Proc Natl Acad Sci. 114, E8711-E8720 (2017).
28. Choy, A., et al., The Legionella effector RavZ inhibits hos autophagy through irreversible Atg8 deconjugation. Science. 338, 1072-6 (2012).
29. Visvikis, O., et al., Innate host defense requires TFEB-mediated transcription of cytoprotective and antimicrobial genes. Immunity. 40, 896-909 (2014).
30. Jacquin, E., et al., Pharmacological modulators of autophagy activate a parallel noncanonical pathway driving unconventional LC3 lipidation. Autophagy. 13, 854-867 (2017).
31. Scotto Rosato, A. S., et al., TRPML1 links lysosomal calcium to autophagosome biogenesis through the activation of the CaMKKβ/VPS34 pathway. Nat Commun. 10, 5630 (2019).
32. Wada, S., et al., The tumor suppressor FLCN mediates an alternate mTOR pathway to regulate browning of adipose tissue. Genes Dev. 30, 2551-2564 (2016).
33. Lawrence, RE, and R Zoncu, The lysosome as a cellular centre for signalling, metabolism and quality control. Nat Cell Bio. 21, 133-142 (2019).
34. Liu, G. Y., and D. M. Sabatini, mTOR at the nexus of nutrition, growth, ageing and disease. Nat Rev Mol Cell Biol. 21, 183-203 (2020).
35. Napolitano, G., et al., A substrate-specific mTORC1 pathway underlies Birt-Hogg-Dube syndrome. Nature (2020).
36. Abu-Remaileh, M., et al., Lysosomal metabolomics reveals vATPase and mTOR-dependent regulation of amino acid efflux from lysosomes. Science. 358, 807-813 (2017).
37. Zhang, H., et al., Mass spectrometry-based carboxyl footprinting of proteins: method evaluation. Int J Mass Spectrom. 312, 78-86 (2012).
38. Kishi-Itakura, C., et al., Ultrastructural analysis of autophagosome organization using mammalian autophagy-deficient cells. J Cell Sci. 127, 4089-4102 (2014).
39. Kishi-Itakura, C., and F., Buss. The use of correlative light-electron microscopy (CLEM) to study PINK1/Parkin-mediated mitophagy. Methods Mol Biol. 1759, 29-39 (2018).
40. McCarthy, D. J., et al., Differential expression analysis of multifactor RNA-seq experiments with respect to biological variation. Nuc Acid Res. 40, 4288-97 (2012).
41. Bouziat, R, et al., Reovirus infection triggers inflammatory responses to dietary antigens and development of celiac disease. Science. 356, 44-50 (2017).
42. Bouziat, R, et al., Murine norovirus infection induces TH1 inflammatory responses to dietary antigens. Cell Host Microbe. 24, 677-688 (2018).
43. DeJesus, et al., R., Functional CRISPR screening identifies the ufmylation pathway as a regulator of SQSTM1/p62. eLife. 5, e17290 (2016).
44. Schmidt, L. S., and W. M. Linehan. FLCN: The causative gene for Birt-Hogg-Dube syndrome. Gene. 640, 28-42 (2018).
45. Perera, R. M., Stoykova, S., Nicolay, B. N., Ross, K. N., Fitament, J., Boukhali, M., Lengrand, J., Deshpande, V., Selig, M. K., Ferrone, C. R., et al. Transcriptional control of autophagy-lysosome function drives pancreatic cancer metabolism. Nature. 524, 361-5 (2015).
46. Gupta, S., Yano, J., Htwe, H. H., Shin, H. R., Cakir, Z., Ituarte, T., Wen, K. W., Kim, G. E., Zoncu, R., Dawson, D. D., and R. M. Perera. Lysosomal retargeting of Myoferlin mitigates membrane stress to enable pancreatic cancer growth. bioRxiv. doi.org/10.1101/2021.01.04.425106 (2021).
47. Nezich, C. L., Wang, C., Fogel, A. I., and R. J. Youle. MiT/TFE3 transcription factors are activated during mitophagy downstream of Parkin and ATG5. J Cell Biol. 210, 435-50 (2015).
48. Heo, J-M., Ordureau, A., Swarup, S., Paulo, J. A., Shen, K., Sabatini, D. M., and J. W. Harper. RAB7A phosphorylation by TBK1 promotes mitophagy via the PINK-PARKIN pathway. Sci Adv. 4, eaav0443 (2018).
49. Lazarou, M., Sliter, D. A., Kane, L. A., Sarraf, S. A., Wang, C., Burman, J. L., Sideris, D. P., Fogel, A. I, and R. J. Youle. The ubiquitin kinase PINK1 recruits autophagy receptors to induce mitophagy. Nature. 524, 309-314 (2015).
50. Birmingham, C. L., Smith, A. C., Bakowski, M. A., Yoshimori, T., and J. H. Brumell. Autophagy controls Salmonella infection in response to damage to the Salmonella-containing vacuole. J Biol Chem. 281, 11374-11383 (2006).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of activating TFEB independent of mTORC1 activity, the method comprising a step of:
contacting a system that comprises:
a membrane comprising LAMP-1, vATPase or GABARAP; and
components of a GABARAP/FLCN/FNIP complex;
with a TRPML1 agonist such that level of the GABARAP/FLCN/FNIP complex at the membrane is elevated.

2. The method of claim 1, wherein the membrane comprising LAMP-1 vATPase or GABARAP defines a compartment.

3. The method of claim 2, wherein the compartment is or comprises a lysosome.

4. The method of claim 1, wherein the membrane is or comprises a lysosomal membrane.

5. The method of claim 5, wherein the lysosomal membrane is part of an intact lysosome.

6. The method of claim 3 or claim 6, wherein the lysosome is in a cell.

7. A method of activating TFEB independent of mTORC1 activity, the method comprising a step of:
administering a TRPML1 agonist.

8. The method of claim 7, wherein the step of administering comprises contacting a system with the TRPML1 agonist, wherein the system comprises:
a lysosomal membrane; and
components of a GABARAP/FLCN/FNIP complex.

9. The method of any one of claims 1-8, wherein the system has a polymorphism or mutation in:
a gene encoding a conjugation machinery protein (conjugation machinery gene) and/or
a gene encoding a component of the GABARAP/FLCN/FNIP complex.

10. The method of claim 9, wherein the conjugation machinery gene is selected from the group consisting of Atg3, Atg5, Atg7, Atg12, Atg16L1, and combinations thereof.

11. The method of claim 10, wherein the conjugation pathway gene is Atg16L1.

12. The method of claim 11, wherein the polymorphism is T300A.

13. The method of any one of claims 1-12, wherein the TRPML1 agonist is of a chemical class selected from the group consisting of polypeptides, nucleic acids, lipids, carbohydrates, small molecules, metals, and combinations thereof.

14. The method of claim 8, wherein the step of administering comprises exposing the system to the TRPML1 agonist under conditions and for a time sufficient that enhanced expression or activity of one or more CLEAR network genes and/or enhancement of one or more of detectable exocytosis activity, autophagy, clearance of lysosomal storage material, and lysosomal biogenesis is observed in the system relative to that prior to the exposure.

15. The method of claim 8, wherein the step of administering comprises exposing the system to the TRPML1 agonist under conditions and for a time sufficient that enhanced expression or activity of one or more genes selected from Table 1 is observed in the system relative to that prior to the exposure.

16. The method of claim 7, wherein the TRPML1 agonist is characterized in that, when assessed for impact on expression of CLEAR network genes, it shows a more restricted impact than that observed under starvation conditions.

17. The method of claim 1 or claim 7, wherein the TRPML1 agonist is characterized in that TRPML1 level or activity is higher in its presence than in its absence, under comparable conditions.

18. The method of claim 1 or claim 7, wherein the TRPML1 agonist is a direct agonist in that it interacts with TRPML1.

19. The method of claim 1 or claim 7, wherein the TRPML1 agonist is an indirect agonist in that it does not directly interact with TRPML1.

20. A method of treating a TRPML1-associated disease, disorder or condition, the method comprising a step of:
administering a TRPML1 agonist to a subject suffering from, or susceptible to, the TRPML1-associated disease, disorder or condition.

21. The method of claim 20, wherein the TRPML1-associated disease, disorder or condition is or comprises an inflammatory condition.

22. The method of claim 20, wherein the TRPML1-associated disease, disorder or condition is or comprises a lysosomal storage disorder.

23. The method of claim 20, wherein the TRPML1-associated disease, disorder or condition is or comprises a polyglutamine disorder.

24. The method of claim 20, wherein the TRPML1-associated disease, disorder or condition is or comprises a neurodegenerative proteinopathy.

25. The method of claim 20, wherein the TRPML1-associated disease, disorder or condition is an infectious disease.

26. The method of claim 20, wherein the TRPML1-associated disease, disorder or condition is selected from a group consisting of Crohn's Disease, Pompe Disease, Parkinson's Disease, Huntington's Disease, Alzheimer's Disease, Spinal-bulbar muscular atrophy, α-1-antitrypsin deficiency, and multiple sulfatase deficiency.

27. The method of claim 20, wherein the TRPML1-associated disease, disorder or condition is Crohn's Disease.

28. A method of activating TFEB by enhancing GABARAP/FNIP/FLCN complex localization at an intracellular membrane surface.

29. The method of claim 28, wherein the intracellular membrane surface is a cytosolic surface of an intracellular compartment.

30. The method of claim 29, wherein the intracellular compartment is a lysosome.

31. The method of claim 29, wherein the intracellular compartment is a mitochondria.

32. The method of claim 29, wherein the intracellular compartment is an endoplasmic reticulum.

33. The method of any one of claims 28-32, wherein the method comprises administering a TRPML1 agonist.

34. The method of any one of claims 28-33, wherein TFEB activation is independent of mTORC1 activity.

35. A method of characterizing a TFEB activating agent, the method comprising:
assessing effect on FLCN localization and/or level of a GABARAP/FNIP/FLCN complex at one or more intracellular membrane surfaces.

36. A method of treating a conjugation-machinery-associated (“CMA”) disease, disorder or condition or a GABARAP/FNIP/FLCN complex-associated disease, disorder or condition, the method comprising a step of:
administering a TRPML1 agonist.

37. The method of claim 27, wherein the disease, disorder or condition is or comprises Crohn's Disease.

38. A method comprising a cellular assay for characterizing activators of TFEB, TFE3 and/or MITF, wherein the cellular assay comprises cells comprising:
(a) presence of a vATPase small molecule inhibitor;
(b) genetic disruption of ATG8 conjugation machinery;
(c) presence of a small molecule inhibitor of ATG8 conjugation machinery;
(d) genetic disruption of a member of a GABARAP subfamily of proteins;
(e) mutation of a LIR domain in FNIP1 or FNIP2;
or a combination thereof.

39. The method of claim 38, wherein the vATPase small molecule inhibitor is Bafilomycin A1.

40. The method of claim 38, wherein the vATPase small molecule inhibitor is not an analogue of Salicylihalamide A.

41. The method of claim 38, wherein the genetic disruption of ATG8 conjugation machinery comprises knock-out of a gene, knock-in of a gene, expression of one or more mutant alleles, siRNA, shRNA, antisense, or a combination thereof.

42. The method of claim 38, wherein the genetic disruption of the member of a GABARAP subfamily of proteins comprises knock-out of a gene, knock-in of a gene, expression of one or more mutant alleles, siRNA, shRNA, antisense, or a combination thereof.
